(19) United States
(12) Patent Application Publication
(10) Pub. No.: US 2025/0088402 A1
(43) Pub. Date: Mar. 13, 2025

(54) SYNCHRONIZED SENSORS AND SYSTEMS

(71) Applicant: QUALCOMM Incorporated, San Diego, CA (US)

(72) Inventors: Ren LI, San Diego, CA (US); John Keith SCHNEIDER, Williamsville, NY (US); Vadim WINEBRAND, San Diego, CA (US); Htet NAING, San Diego, CA (US)

(21) Appl. No.: 18/466,721

(22) Filed: Sep. 13, 2023

(51) Int. Cl. H04L 27/26; A61B 5/00; A61B 5/021

(52) U.S. Cl. CPC H04L 27/2655; A61B 5/0004; A61B 5/002; A61B 5/0024; A61B 5/02125

(57) ABSTRACT

Synchronized sensors and systems are disclosed. Techniques involving a synchronized sensor system for determining a physiological parameter of a user may include: obtaining one or more first measurements at a first location of the user via a first sensor; obtaining one or more second measurements at a second location of the user via a second sensor; and determining the physiological parameter of the user based on the one or more first measurements, the one or more second measurements, and a distance between the first location and the second location, the distance between the first location and the second location determined based on acoustic communication between the first sensor and the second sensor. In some implementations, acoustic communication may include ultrasound signals between the first sensor and the second sensor, which may be time synchronized by exchanging timestamps.

### TECHNICAL FIELD

This disclosure relates generally to devices and systems using multiple types of sensors.

### DESCRIPTION OF RELATED TECHNOLOGY

A variety of different sensing technologies and algorithms are being implemented in devices for various biometric and biomedical applications, including health and wellness monitoring. This push is partly a result of the limitations in the usability of traditional measuring devices for continuous, noninvasive and/or ambulatory monitoring. Some such devices are, or include, photoacoustic sensors or optical sensors. Although some previously deployed devices can provide acceptable results, improved detection devices and systems would be desirable.

### SUMMARY

The systems, methods and devices of this disclosure each have several aspects, no single one of which is solely responsible for the desirable attributes disclosed herein.

In one aspect of the present disclosure, a synchronized sensor system is disclosed. In some embodiments, the synchronized sensor system may include: a first sensor disposed at a first location of a user and configured to obtain first measurements associated with the user at the first location; and a second sensor disposed at a second location of the user and configured to obtain second measurements associated with the user at the second location, the second sensor configured to perform acoustic communication with the first sensor and radio frequency (RF) data communication with the first sensor, a host device, or a combination thereof.

In one variant thereof, at least a portion of the acoustic communication with the first sensor may enable determination, by the second sensor, of a distance between the first location and the second location.

In another variant thereof, at least a portion of the RF data communication may enable determination, by the second sensor or the host device, of a physiological parameter associated with the user based on the first measurements associated with the user at the first location, the second measurements associated with the user at the second location, and the distance between the first location and the second location.

In another aspect of the present disclosure, a method of determining a physiological parameter of a user using synchronized sensors is disclosed. In some embodiments, the method may include: obtaining one or more first measurements at a first location of the user via a first sensor; obtaining one or more second measurements at a second location of the user via a second sensor; and determining the physiological parameter of the user based on the one or more first measurements, the one or more second measurements, and a distance between the first location and the second location, the distance between the first location and the second location determined based on acoustic communication between the first sensor and the second sensor.

In another aspect of the present disclosure, an apparatus is disclosed. In some embodiments, the apparatus may include: means for obtaining one or more first measurements at a first location of a user via a first sensor; means for obtaining one or more second measurements at a second location of the user via a second sensor; and means for determining a physiological parameter of the user based on the one or more first measurements, the one or more second measurements, and a distance between the first location and the second location, the distance between the first location and the second location determined based on acoustic communication between the first sensor and the second sensor.

In another aspect of the present disclosure, a non-transitory computer-readable apparatus is disclosed. In some embodiments, the non-transitory computer-readable apparatus may include a storage medium, the storage medium including a plurality of instructions configured to, when executed by one or more processors, cause an apparatus to: obtain one or more first measurements at a first location of a user via a first sensor; obtain one or more second measurements at a second location of the user via a second sensor; and determine a physiological parameter of the user based on the one or more first measurements, the one or more second measurements, and a distance between the first location and the second location, the distance between the first location and the second location determined based on acoustic communication between the first sensor and the second sensor.

Details of one or more implementations of the subject matter described in this disclosure are set forth in the accompanying drawings and the description below. Other features, aspects, and advantages will become apparent from the description, the drawings and the claims. Note that the relative dimensions of the following figures may not be drawn to scale.

### BRIEF DESCRIPTION OF THE DRAWINGS

FIG. 1 shows an example of a blood pressure monitoring device based on photoacoustic plethysmography, which may be referred to herein as PAPG.

FIG. 2 shows an example of a blood pressure monitoring device based on photoplethysmography (PPG).

FIG. 3 is a block diagram that shows example components of a photoacoustic sensor system according to some disclosed implementations.

FIG. 4 is a block diagram that shows example components of a sensor apparatus according to some implementations.

FIG. 4A illustrates an example encoding by an ultrasound transmitter of the sensor apparatus of FIG. 4.

FIG. 4B illustrates an example decoding by the ultrasound receiver of the sensor apparatus of FIG. 4.

FIG. 5 is a block diagram that shows example components of a sensor apparatus according to some implementations.

FIG. 5A illustrates an example of a potential occurring via electrocardiogram (EKG) leads.

FIGS. 5B and 5C illustrate examples of possible positions of the sensor apparatus of FIG. 5 on a user's limbs.

FIG. 6 shows examples of heart rate waveform (HRW) features that may be extracted according to some implementations.

FIG. 7 shows examples of devices that may be used in a system for estimating blood pressure based, at least in part, on pulse transit time (PTT).

FIG. 8 shows a cross-sectional side view of a diagrammatic representation of a portion of an artery through which a pulse is propagating.

FIG. 9A shows an example monitoring device designed to be worn around a wrist according to some implementations.

FIG. 9B shows an example monitoring device designed to be worn on a finger according to some implementations.

FIG. 9C shows an example monitoring device designed to reside on an earbud according to some implementations.

FIG. 10 is a diagram showing an example of sensor apparatus coupled physically.

FIG. 11 is a diagram showing an example of sensor apparatus coupled and synchronized wirelessly.

FIG. 12 is a block diagram that shows an example configuration of sensors wirelessly synchronized with a host device according to some implementations.

FIG. 13 shows an example time synchronization via timestamp exchange according to some implementations.

FIG. 13A is a call flow diagram representing an example two-way exchange between a first clock timer of a first sensor and a second clock timer of a second sensor.

FIGS. 14A and 14B are block diagrams that show another example configuration of sensors wirelessly synchronized with a host device according to some implementations.

FIG. 15A-15D illustrate various examples of distance measurements through tissue between two sensors using acoustic measurements according to some implementations.

FIG. 16 is a flow diagram of a method for determining a physiological parameter of a user, according to some disclosed implementations.

Like reference numbers and designations in the various drawings indicate like elements.

### DETAILED DESCRIPTION

As used herein, an “RF signal” comprises an electromagnetic wave that transports information through the space between a transmitter (or transmitting device) and a receiver (or receiving device). As used herein, a transmitter may transmit a single “RF signal” or multiple “RF signals” to a receiver. However, the receiver may receive multiple “RF signals” corresponding to each transmitted RF signal due to the propagation characteristics of RF signals through multiple channels or paths.

The following description is directed to certain implementations for the purposes of describing various aspects of this disclosure. However, a person having ordinary skill in the art will readily recognize that the teachings herein can be applied in a multitude of different ways. Some of the concepts and examples provided in this disclosure are especially applicable to blood pressure monitoring applications or monitoring of other physiological parameters. However, some implementations also may be applicable to other types of biological sensing applications, as well as to other fluid flow systems. The described implementations may be implemented in any device, apparatus, or system that includes an apparatus as disclosed herein. In addition, it is contemplated that the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands, patches, chest bands, anklets, etc., Bluetooth® devices, personal data assistants (PDAs), wireless electronic mail receivers, hand-held or portable computers, netbooks, notebooks, smartbooks, tablets, printers, copiers, scanners, facsimile devices, global positioning system (GPS) receivers/navigators, cameras, digital media players, game consoles, wrist watches, clocks, calculators, television monitors, flat panel displays, electronic reading devices (e.g., e-readers), mobile health devices, computer monitors, auto displays (including odometer and speedometer displays, etc.), cockpit controls and/or displays, camera view displays (such as the display of a rear view camera in a vehicle), architectural structures, microwaves, refrigerators, stereo systems, cassette recorders or players, DVD players, CD players, VCRs, radios, portable memory chips, washers, dryers, washer/dryers, parking meters, automobile doors, autonomous or semi-autonomous vehicles, drones, Internet of Things (IoT) devices, etc. Thus, the teachings are not intended to be limited to the specific implementations depicted and described with reference to the drawings; rather, the teachings have wide applicability as will be readily apparent to persons having ordinary skill in the art.

There is a strong need for accurate, non-invasive, continuous monitoring wearable devices for both clinical and consumer applications, e.g., for measuring physiological parameters such as blood pressure of a user. In particular, non-invasive monitoring of blood pressure is desirable. Continuous blood pressure monitoring opens avenues for efficient and effective diagnosis and treatment of cardiovascular conditions (e.g., hypertension), cardiovascular event detection, and stress monitoring. It would also allow daily spot checks of cardiovascular conditions including blood pressure, as well as overnight sleep monitoring.

Flexible configurations of sensing mechanisms that allow biometric collection and physiological characteristics related to blood pressure estimation, such as pulse transmit time (PTT), pulse wave velocity (PWV) of a blood vessel, and artery diameter and distension, could be a step in that direction. PTT and PWV are important characteristics that are a function of the arterial wall stiffness and tension, blood density, body posture, blood pressure, and more. It would thus be valuable to obtain such characteristics with accuracy and convenience.

Multiple sensors configured to obtain different types of measurements, such as photoacoustic measurements, optical measurements, bioimpedance measurements, ultrasound probes, and/or EKG, can be used to derive PTT, PWV, and other cardiac features. However, PWV accuracy can be affected by a distance between sensors. Current sensors such as EKG sensors are connected by wire for data acquisition and synchronicity, which results in a bulky deployment not conducive to user convenience. Moreover, two sensors can be difficult to align with a given artery to get good signals simultaneously, as it may require an additional offset adjustment mechanism.

Another limitation of previously deployed multiple-sensor implementations is that the multiple sensors typically have to be placed on the same side of the body (left or right). Otherwise, an incorrect PWV can be derived when placing one sensor (e.g., photoacoustic sensor) on one side of the body (e.g., left wrist) and another sensor (e.g., photoacoustic sensor) on the other side of the body (e.g., right wrist). In such a scenario, the distance obtained from ultrasound waves sent over the air for direct distance measurement may not be correlated to the actual pulse wave traveling along an artery within the body. This error may exist as long as the pulse wave traveling distance along the artery between sensors is deviated from the direct over-the-air distance. Thus, measured sensor distances are not accurate, and there is a need to improve distance estimation accuracy and provide flexibility to allow multiple multi-modal PWV measurement (using sensors not necessarily of identical modality) at different locations of body (wrist, finger, chest, etc.) simultaneously.

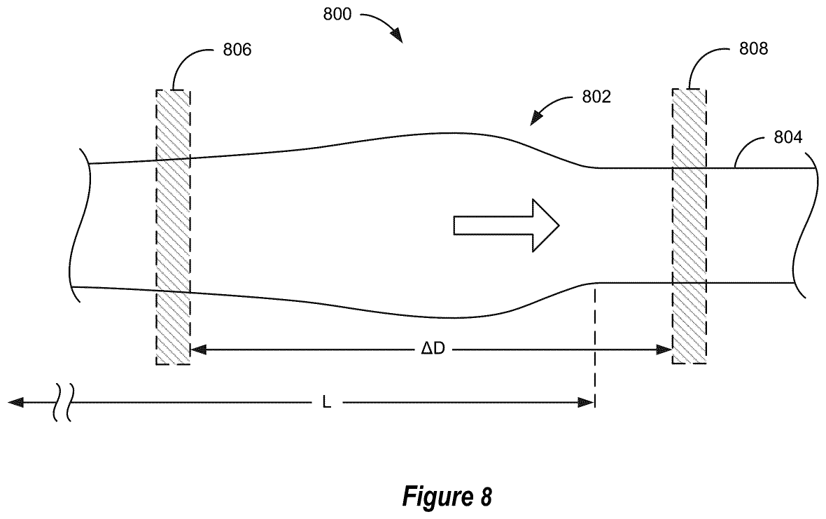

Various aspects provided in the present disclosure relate generally to an approach and a system that uses multiple sensors that use different sensing modalities to determine accurate physiological characteristics of the cardiovascular system (e.g., PTT and PWV) and thereby accurately estimate a physiological parameter of the user (including, e.g., blood pressure). Some aspects more specifically relate to a synchronized sensor system configured to obtain at least first measurements at a first location and second measurements at a second location of a user's body. In some configurations, third measurements and more may also be obtained with at least another, third sensor. Such measurements may be optical, photoacoustic, electrical (e.g., electrocardiogram), or combinations thereof. In some examples, the sensors each may be capable of two types of wireless communication including radio frequency (RF) signaling and ultrasound-based communication. In some such examples, RF signaling can be used to synchronize the clock timers of the sensors by exchanging timestamps. In some implementations, synchronization may occur with a host device (e.g., user device such as smartphone or wristwatch) via RF links, and measurements can be sent to the host device. In some implementations, a primary sensor can estimate the characteristic and parameters based on measurements obtained at the primary sensor and other secondary sensor(s), and the host device may retrieve the estimations from the primary sensor via an RF link. Acoustic (e.g., ultrasound) signals from two or more sensors having ultrasound transceivers can be used to determine a distance between two given sensors, where the ultrasound signals can travel through tissue. By acquiring a time-synchronized estimation of the time of flight based on the speed of sound through tissue (or another medium such as air in appropriate cases), distance between the two locations can be estimated. PWV can also be estimated based on the measured distance and time. In turn, blood pressure can be estimated based on the PWV, as discussed with respect to FIG. 8.

Further, in some implementations, machine learning can be used to train a machine learning model that can predict a physiological characteristic of a blood vessel (e.g., PTT, PWV) or parameter of a user (e.g., blood pressure), or examine such a physiological characteristic or parameter estimated using the sensor or host device. Based on any discrepancies between the sensor-based estimation and the model-generated prediction, some or all of the sensor-based measurements can be kept or discarded.

Particular implementations of the subject matter described in this disclosure can be implemented to realize one or more of the following potential advantages. Synchronization of sensors can enable a flexible setup and implementation of the sensors at any part of the body. In some examples, the sensors are compact, wireless, and wearable, and can be flexibly placed and set up in almost any part of the body-on the wrist, ankle, waist, neck, etc. allowing the sensors to be used in a user-friendly and convenient manner. RF and ultrasound signals between sensor nodes are robust to occlusions. Wireless synchronization between sensors can reduce clock drift. More specifically, timestamp messaging between sensors can result in virtually no time sync error (under 1 microsecond) based on careful timing offset control, drift or skew estimation and compensation, or combinations thereof. Some implementations are configured for wireless communication via three different data communication methods among sensors and host devices: RF (e.g., Bluetooth), ultrasound over the air, and ultrasound through tissue. Wireless synchronization with a host device can further reduce the size of the sensor, as the host device can offload computational burden in some implementations by obtaining the measurements from the sensors and deriving the PWV and blood pressure. In some implementations, a primary sensor can handle computational and memory load associated with itself and secondary sensors. Having a central device and wirelessly connected peripheral devices can result in a simplified topography of sensors where one device can handle computations based on measurements from all sensors, which can result in better power efficiency. In either implementation, each sensor obtains measurements at their respective locations in a synchronized manner.

Additional details will follow after an initial description of relevant systems and technologies.

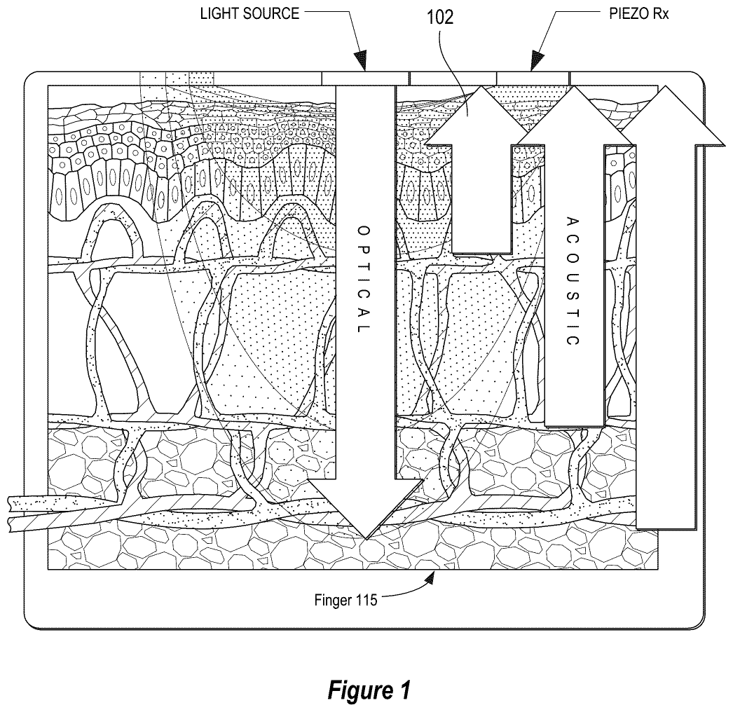

FIG. 1 shows an example of a blood pressure monitoring device based on photoacoustic plethysmography, which is referred to herein as PAPG. FIG. 1 shows the same examples of arteries, veins, arterioles, venules and capillaries inside a body part, which is a finger 115 in this example. In some examples, the light source shown in FIG. 1 may be coupled to a light source system (not shown) that is disposed remotely from the body part (e.g., finger 115). In some implementations, the light source may be an opening of an optical fiber or other waveguide. Such an opening may also be connected to an opening of an interface that is contactable with the body part. In some embodiments, the light source system may include one or more LEDs, one or more laser diodes, etc. In this example, the light source has transmitted light (in some examples, green, red, infrared, and/or near-infrared (NIR) light) that has penetrated the tissues of the finger 115 in an illuminated zone.

In the example shown in FIG. 1, blood vessels (and components of the blood itself) are heated by the incident light from the light source and are emitting acoustic waves 102. In this example, the emitted acoustic waves 102 include ultrasonic waves. According to this implementation, the acoustic wave emissions 102 are being detected by an ultrasonic receiver, which is a piezoelectric receiver in this example. Photoacoustic emissions 102 from the illuminated tissues, detected by the piezoelectric receiver, may be used to detect volumetric changes in the blood of the illuminated zone of the finger 115 that correspond to physiological data within the illuminated tissues of finger 115, such as heart rate waveforms. Although some of the tissue areas shown to be illuminated are offset from those shown to be producing photoacoustic emissions 102, this is merely for illustrative convenience. It will be appreciated that that the illuminated tissues will actually be those producing photoacoustic emissions. Moreover, it will be appreciated that the maximum levels of photoacoustic emissions will often be produced along the same axis as the maximum levels of illumination.

One important difference between an optical technique such as a photoplethysmography (PPG)-based system the PAPG-based method of FIG. 1 is that the acoustic waves shown in FIG. 1 travel much more slowly than the reflected light waves involved in PPG. Accordingly, depth discrimination based on the arrival times of the acoustic waves shown in FIG. 1 is possible, whereas depth discrimination based on the arrival times of the light waves in PPG may not be possible. This depth discrimination allows some disclosed implementations to isolate acoustic waves received from the different blood vessels.

According to some such examples, such depth discrimination allows artery heart rate waveforms to be distinguished from vein heart rate waveforms and other heart rate waveforms. Therefore, blood pressure estimation based on depth-discriminated PAPG methods can be substantially more accurate than blood pressure estimation based on PPG-based methods.

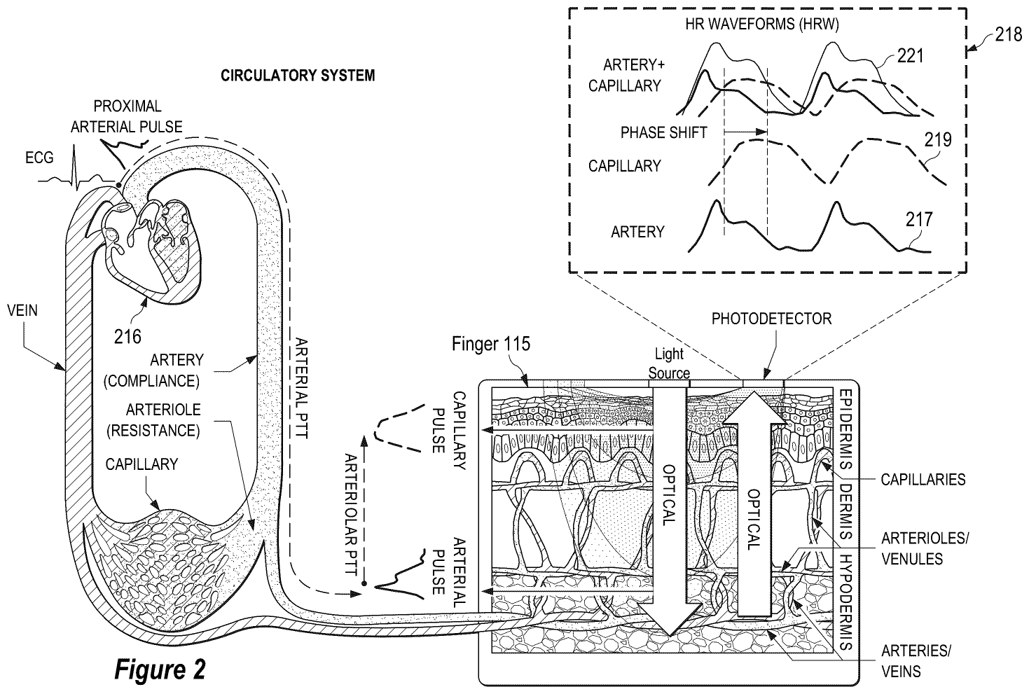

FIG. 2 shows an example of a blood pressure monitoring device based on photoplethysmography (PPG). FIG. 2 shows examples of arteries, veins, arterioles, venules and capillaries of a circulatory system, including those inside a finger 115. In the example shown in FIG. 2, an electrocardiogram (EKG) sensor has detected a proximal arterial pulse near the heart 216. Some examples are described below of measurement of the arterial pulse transit time (PTT) according to arterial pulses measured by two sensors, one of which may be an electrocardiogram sensor in some implementations.

According to the example shown in FIG. 2, a light source that includes one or more lasers or light-emitting diodes (LEDs) has transmitted light (in some examples, green, red, infrared, and/or near-infrared (NIR) light) that has penetrated the tissues of the finger 115 in an illuminated zone. Reflections from these tissues, detected by a photodetector, may be used to detect volumetric changes in the blood of the illuminated zone of the finger 115 that correspond to heart rate waveforms.

As shown in the heart rate waveform graphs 218 of FIG. 2, the capillary heart rate waveform 219 is differently-shaped and phase-shifted relative to the artery heart rate waveform 217. In this simple example, the detected heart rate waveform 221 is a combination of the capillary heart rate waveform 219 and the artery heart rate waveform 217. In some instances, the responses of one or more other blood vessels may also be part of the heart rate waveform 221 detected by a PPG-based blood pressure monitoring device.

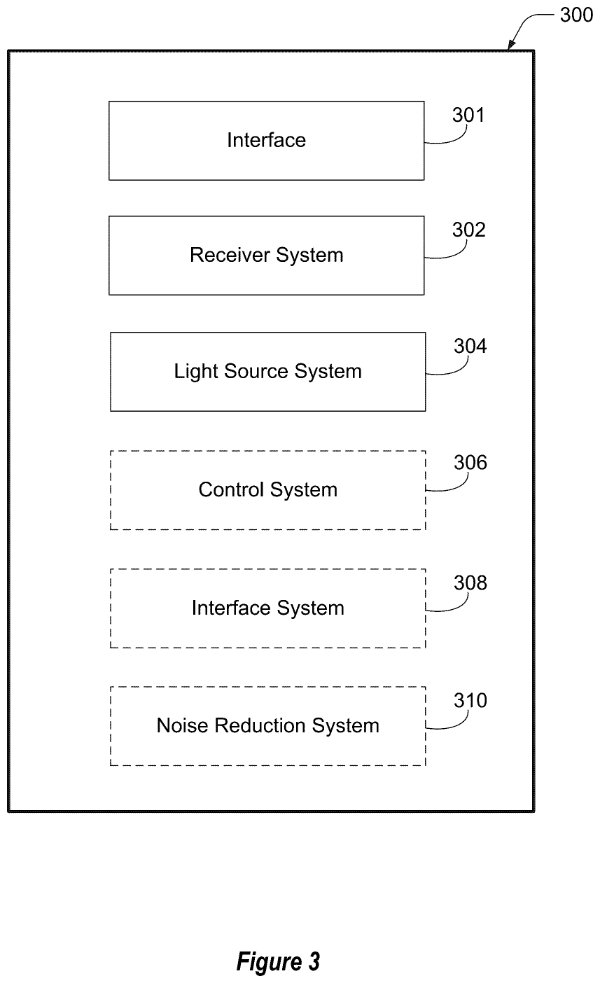

FIG. 3 is a block diagram that shows example components of a photoacoustic sensor system 300 according to some disclosed implementations. In this example, the photoacoustic sensor system 300 includes an interface 301, a receiver system 302, and a light source system 304. In some cases, a waveguide system may be included as a separate part of the photoacoustic sensor system 300, or in some cases, may be part of the light source system 304. Some implementations of the photoacoustic sensor system 300 may include a control system 306, an interface system 308, a noise reduction system 310, or a combination thereof.

Various examples of the interface 301 and various configurations of light source systems 304 and receiver systems 302 are disclosed herein. Some examples are described in more detail below.

Some disclosed PAPG sensors described herein (such as photoacoustic sensor system 300) may include a platen, a light source system, and an ultrasonic receiver system. According to some implementations, the light source system may include a light source configured to produce and direct light. In some implementations, the platen may include an anti-reflective layer, a mirror layer, or combinations thereof. According to some implementations, the platen may have an outer surface, or a layer on the outer surface, with an acoustic impedance that is configured to approximate the acoustic impedance of human skin. In some implementations, the platen may have a surface proximate the ultrasonic receiver system, or a layer on the surface proximate the ultrasonic receiver system, with an acoustic impedance that is configured to approximate the acoustic impedance of the ultrasonic receiver system.

Some disclosed PAPG sensors described herein (such as photoacoustic sensor system 300) may include an interface, a light source system and an ultrasonic receiver system. Some such devices may not include a rigid platen. According to some implementations, the interface may be a physical, flexible interface constructed of one or more of suitable materials having a desired property or properties (e.g., an acoustic property such as acoustic impedance, softness of the material). In some implementations, the interface may be a flexible interface that can contact a target object that may be proximate to or contact the interface. There may be salient differences between such an interface and a platen. In some implementations, the light source system may be configured to direct light using one or more optical waveguides (e.g., optical fibers) configured to direct light toward a target object. According to some implementations, the interface may have an outer surface, or a layer on the outer surface, with an acoustic impedance that is configured to approximate the acoustic impedance of human skin. Such outer surface may have a contact portion that is contactable by a user or a body part of the user (e.g., finger, wrist). In some examples, the optical waveguide(s) may be embedded in one or more acoustic matching layers that are configured to bring the light transmitted by the optical waveguide(s) very close to tissue. The outer surface and/or other parts of the interface may be compliant, pliable, flexible, or otherwise at least partially conforming to the shape and contours of the body part of the user. In some implementations, the interface may have a surface proximate the ultrasonic receiver system, or a layer on the surface proximate the ultrasonic receiver system, with an acoustic impedance that is configured to approximate the acoustic impedance of the ultrasonic receiver system.

In some implementations in which the receiver system 302 includes an ultrasonic receiver system, the interface 301 may be an interface having a contact portion configured to make contact with a body part of a user such as the finger 115 shown in FIG. 1.

In some embodiments, the light source system 304 may, include one or more one or more light sources. In some implementations, the light source system 304 may include one or more light-emitting diodes. In some implementations, the light source system 304 may include one or more laser diodes. According to some implementations, the light source system 304 may include one or more vertical-cavity surface-emitting lasers (VCSELs). In some implementations, the light source system 304 may include one or more edge-emitting lasers. In some implementations, the light source system 304 may include one or more neodymium-doped yttrium aluminum garnet (Nd:YAG) lasers.

Hence, the light source system 304 may include, for example, a laser diode, a light-emitting diode (LED), or an array of either or both. The light source system 304 may be configured to generate and emit optical signals. The light source system 304 may, in some examples, be configured to transmit light in one or more wavelength ranges. In some examples, the light source system 304 may be configured to transmit light in a wavelength range of 500 to 600 nanometers (nm). According to some examples, the light source system 304 may be configured to transmit light in a wavelength range of 800 to 950 nm. According to some examples, the light source system 304 may be configured to transmit light in infrared or near infrared (NIR) region of the electromagnetic spectrum (about 700 to 2500 nm). In view of factors such as skin reflectance, fluence, the absorption coefficients of blood and various tissues, and skin safety limits, one or both of these wavelength ranges may be suitable for various use cases. For example, the wavelength ranges of 500 nm to 600 nm and of 800 to 950 nm may both be suitable for obtaining photoacoustic responses from relatively smaller, shallower blood vessels, such as blood vessels having diameters of approximately 0.5 mm and depths in the range of 0.5 mm to 1.5 mm, such as may be found in a finger. The wavelength range of 800 to 950 nm, or about 700 to 900 nm, or about 600 to 1100 nm may, for example, be suitable for obtaining photoacoustic responses from relatively larger, deeper blood vessels, such as blood vessels having diameters of approximately 2.0 mm and depths in the range of 2 mm to 3 mm, such as may be found in an adult wrist. In some implementations, the light source system 304 may be configured to switch wavelengths to capture acoustic information from different depths, e.g., based on signal(s) from the control system 306.

In some implementations, the light source system 304 may be configured for emitting various wavelengths of light, which may be selectable to trigger acoustic wave emissions primarily from a particular type of material. For example, because the hemoglobin in blood absorbs near-infrared light very strongly, in some implementations the light source system 304 may be configured for emitting one or more wavelengths of light in the near-infrared range, in order to trigger acoustic wave emissions from hemoglobin. However, in some examples, the control system 306 may control the wavelength(s) of light emitted by the light source system 304 to preferentially induce acoustic waves in blood vessels, other soft tissue, and/or bones. For example, an infrared (IR) light-emitting diode LED may be selected and a short pulse of IR light emitted to illuminate a portion of a target object and generate acoustic wave emissions that are then detected by the receiver system 302. In another example, an IR LED and a red LED or other color such as green, blue, white or ultraviolet (UV) may be selected and a short pulse of light emitted from each light source in turn with ultrasonic images obtained after light has been emitted from each light source. In other implementations, one or more light sources of different wavelengths may be fired in turn or simultaneously to generate acoustic emissions that may be detected by the ultrasonic receiver. Image data from the ultrasonic receiver that is obtained with light sources of different wavelengths and at different depths (e.g., varying range gate delays (RGDs)) into the target object may be combined to determine the location and type of material in the target object. Image contrast may occur as materials in the body generally absorb light at different wavelengths differently. As materials in the body absorb light at a specific wavelength, they may heat differentially and generate acoustic wave emissions with sufficiently short pulses of light having sufficient intensities. Depth contrast may be obtained with light of different wavelengths and/or intensities at each selected wavelength. That is, successive images may be obtained at a fixed RGD (which may correspond with a fixed depth into the target object) with varying light intensities and wavelengths to detect materials and their locations within a target object. For example, hemoglobin, blood glucose or blood oxygen within a blood vessel inside a target object such as a finger may be detected photoacoustically.

According to some implementations, the light source system 304 may be configured for emitting a light pulse with a pulse width less than about 100 nanoseconds. In some implementations, the light pulse may have a pulse width between about 10 nanoseconds and about 500 nanoseconds or more. According to some examples, the light source system 304 may be configured for emitting a plurality of light pulses at a pulse repetition frequency between 10 Hz and 100 kHz. Alternatively, or additionally, in some implementations the light source system 304 may be configured for emitting a plurality of light pulses at a pulse repetition frequency between about 1 MHz and about 100 MHz. Alternatively, or additionally, in some implementations the light source system 304 may be configured for emitting a plurality of light pulses at a pulse repetition frequency between about 10 Hz and about 1 MHz. In some examples, the pulse repetition frequency of the light pulses may correspond to an acoustic resonant frequency of the ultrasonic receiver and the substrate. For example, a set of four or more light pulses may be emitted from the light source system 304 at a frequency that corresponds with the resonant frequency of a resonant acoustic cavity in the sensor stack, allowing a build-up of the received ultrasonic waves and a higher resultant signal strength. In some implementations, filtered light or light sources with specific wavelengths for detecting selected materials may be included with the light source system 304. In some implementations, the light source system 304 may contain light sources such as red, green and blue LEDs of a display that may be augmented with light sources of other wavelengths (such as IR and/or UV) and with light sources of higher optical power. For example, high-power laser diodes or electronic flash units (e.g., an LED or xenon flash unit) with or without filters may be used for short-term illumination of the target object.

According to some examples, the light source system 304 may also include one or more light-directing elements configured to direct light from the light source system 304 towards the target object along the first axis. In some examples, the one or more light-directing elements may include at least one diffraction grating. Alternatively, or additionally, the one or more light-directing elements may include at least one lens.

In various configurations, the light source system 304 may incorporate anti-reflection (AR) coating, a mirror, a light-blocking layer, a shield to minimize crosstalk, etc.

The light source system 304 may include various types of drive circuitry, depending on the particular implementation. In some disclosed implementations, the light source system 304 may include at least one multi-junction laser diode, which may produce less noise than single-junction laser diodes. In some examples, the light source system 304 may include a drive circuit (also referred to herein as drive circuitry) configured to cause the light source system 304 to emit pulses of light at pulse widths in a range from 3 nanoseconds to 1000 nanoseconds. According to some examples, the light source system 304 may include a drive circuit configured to cause the light source system 304 to emit pulses of light at pulse repetition frequencies in a range from 1 kilohertz to 100 kilohertz.

In some example implementations, some or all of the one or more light sources of the light source system 304 may be disposed at or along an axis that is parallel to or angled relative to a central axis associated with the interface or platen 301. Optical signals may be emitted toward a target object (e.g., blood vessel), which may cause generation of ultrasonic waves by the target object. These ultrasonic waves may be detectable by one or more receiver elements of a receiver system 302.

Various examples of a receiver system 302 are disclosed herein, some of which may include ultrasonic receiver systems, optical receiver systems, or combinations thereof. In some implementations, the receiver system 302 includes an ultrasonic receiver system having the one or more receiver elements. In implementations that include an ultrasonic receiver system, the ultrasonic receiver and an ultrasonic transmitter may be combined in an ultrasonic transceiver. In some examples, the receiver system 302 may include a piezoelectric receiver layer, such as a layer of PVDF polymer or a layer of PVDF-TrFE copolymer. In some implementations, a single piezoelectric layer may serve as an ultrasonic receiver. In some implementations, other piezoelectric materials may be used in the piezoelectric layer, such as aluminum nitride (AlN) or lead zirconate titanate (PZT). The receiver system 302 may, in some examples, include an array of ultrasonic transducer elements, such as an array of piezoelectric micromachined ultrasonic transducers (PMUTs), an array of capacitive micromachined ultrasonic transducers (CMUTs), etc. In some such examples, a piezoelectric receiver layer, PMUT elements in a single-layer array of PMUTs, or CMUT elements in a single-layer array of CMUTs, may be used as ultrasonic transmitters as well as ultrasonic receivers. According to some examples, the receiver system 302 may be, or may include, an ultrasonic receiver array. In some examples, the sensor apparatus 400 may include one or more separate ultrasonic transmitter elements or one or more separate arrays of ultrasonic transmitter elements. In some examples, the ultrasonic transmitter(s) may include an ultrasonic plane-wave generator.

In some implementations, at least portions of the photoacoustic sensor system 300 (for example, the receiver system 302, the light source system 304, or both) may include one or more sound-absorbing layers, acoustic isolation material, light-absorbing material, light-reflecting material, or combinations thereof. In some examples, acoustic isolation material may reside between the light source system 304 and at least a portion of the receiver system 302. In some examples, at least portions of the photoacoustic sensor system 300 (for example, the receiver system 302, the light source system 304, or both) may include one or more electromagnetically shielded transmission wires. In some such examples, the one or more electromagnetically shielded transmission wires may be configured to reduce electromagnetic interference from the light source system 304 that is received by the receiver system 302.

The control system 306 may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system 306 also may include (and/or be configured for communication with) one or more memory devices, such as one or more random access memory (RAM) devices, read-only memory (ROM) devices, etc. Accordingly, the photoacoustic sensor system 300 may have a memory system that includes one or more memory devices, though the memory system is not shown in FIG. 3. The control system 306 may be configured for receiving and processing data from the receiver system 302, e.g., as described below. If the photoacoustic sensor system 300 includes an ultrasonic transmitter, the control system 306 may be configured for controlling the ultrasonic transmitter. In some implementations, functionality of the control system 306 may be partitioned between one or more controllers or processors, such as a dedicated sensor controller and an applications processor of a mobile device.

In some examples, the control system 306 may be communicatively coupled to the light source system 304 and configured to control the light source system to emit light towards a target object on an outer surface of the interface 301. In some such examples, the control system 306 may be configured to receive signals from the ultrasonic receiver system (including one or more receiver elements) corresponding to the ultrasonic waves generated by the target object responsive to the light from the light source system. In some examples, the control system 306 may be configured to identify one or more blood vessel signals, such as arterial signals or vein signals, from the ultrasonic receiver system. In some such examples, the one or more arterial signals or vein signals may be, or may include, one or more blood vessel wall signals corresponding to ultrasonic waves generated by one or more arterial walls or vein walls of the target object. In some such examples, the one or more arterial signals or vein signals may be, or may include, one or more arterial blood signals corresponding to ultrasonic waves generated by blood within an artery of the target object or one or more vein blood signals corresponding to ultrasonic waves generated by blood within a vein of the target object. In some examples, the control system 306 may be configured to determine or estimate one or more physiological parameters or cardiac features based, at least in part, on one or more arterial signals, on one or more vein signals, or on combinations thereof. According to some examples, a physiological parameter may be, or may include, blood pressure. In some approaches, blood pressure can be estimated based at least on PWV, as will be discussed below with respect to FIG. 8.

In further examples, the control system 306 may be communicatively coupled to the receiver system 302. The receiver system 302 may be configured to detect acoustic signals from the target object. The control system 306 may be configured to select at least one of a plurality of receiver elements of the receiver system 302. Such selected receiver element(s) may correspond to the best signals from multiple receiver elements. In some embodiments, the selection of the at least one receiver element may be based on information regarding detected acoustic signals (e.g., arterial signals or vein signals) from the plurality of receivers. For example, signal quality or signal strength (based, e.g., on signal-to-noise ratio (SNR)) of some signals may be relatively higher than some others or above a prescribed threshold or percentile, which may indicate the best signals. In some implementations, the control system 306 may also be configured to, based on the information regarding detected acoustic signals, determine or estimate at least one characteristic of the blood vessels such as PWV (indicative of arterial stiffness), arterial dimensions, or both.

Some implementations of the photoacoustic sensor system 300 may include an interface system 308. In some examples, the interface system 308 may include a wireless interface system. In some implementations, the interface system 408 may include a user interface system, one or more network interfaces, one or more interfaces between the control system 306 and a memory system and/or one or more interfaces between the control system 306 and one or more external device interfaces (e.g., ports or applications processors), or combinations thereof. According to some examples in which the interface system 308 is present and includes a user interface system, the user interface system may include a microphone system, a loudspeaker system, a haptic feedback system, a voice command system, one or more displays, or combinations thereof. According to some examples, the interface system 308 may include a touch sensor system, a gesture sensor system, or a combination thereof. The touch sensor system (if present) may be, or may include, a resistive touch sensor system, a surface capacitive touch sensor system, a projected capacitive touch sensor system, a surface acoustic wave touch sensor system, an infrared touch sensor system, any other suitable type of touch sensor system, or combinations thereof.

In some examples, the interface system 308 may include a force sensor system. The force sensor system (if present) may be, or may include, a piezo-resistive sensor, a capacitive sensor, a thin film sensor (for example, a polymer-based thin film sensor), another type of suitable force sensor, or combinations thereof. If the force sensor system includes a piezo-resistive sensor, the piezo-resistive sensor may include silicon, metal, polysilicon, glass, or combinations thereof. An ultrasonic fingerprint sensor and a force sensor system may, in some implementations, be mechanically coupled. In some implementations, the force sensor system may be mechanically coupled to a platen. In some such examples, the force sensor system may be integrated into circuitry of the ultrasonic fingerprint sensor. In some examples, the interface system 308 may include an optical sensor system, one or more cameras, or a combination thereof.

According to some examples, the photoacoustic sensor system 300 may include a noise reduction system 310. For example, the noise reduction system 310 may include one or more mirrors that are configured to reflect light from the light source system 304 away from the receiver system 302. In some implementations, the noise reduction system 310 may include one or more sound-absorbing layers, acoustic isolation material, light-absorbing material, light-reflecting material, or combinations thereof. In some examples, the noise reduction system 310 may include acoustic isolation material, which may reside between the light source system 304 and at least a portion of the receiver system 302, on at least a portion of the receiver system 302, or combinations thereof. In some examples, the noise reduction system 310 may include one or more electromagnetically shielded transmission wires. In some such examples, the one or more electromagnetically shielded transmission wires may be configured to reduce electromagnetic interference from circuitry of the light source system, receiver system circuitry, or combinations thereof, that is received by the receiver system.

### Example Sensor Systems

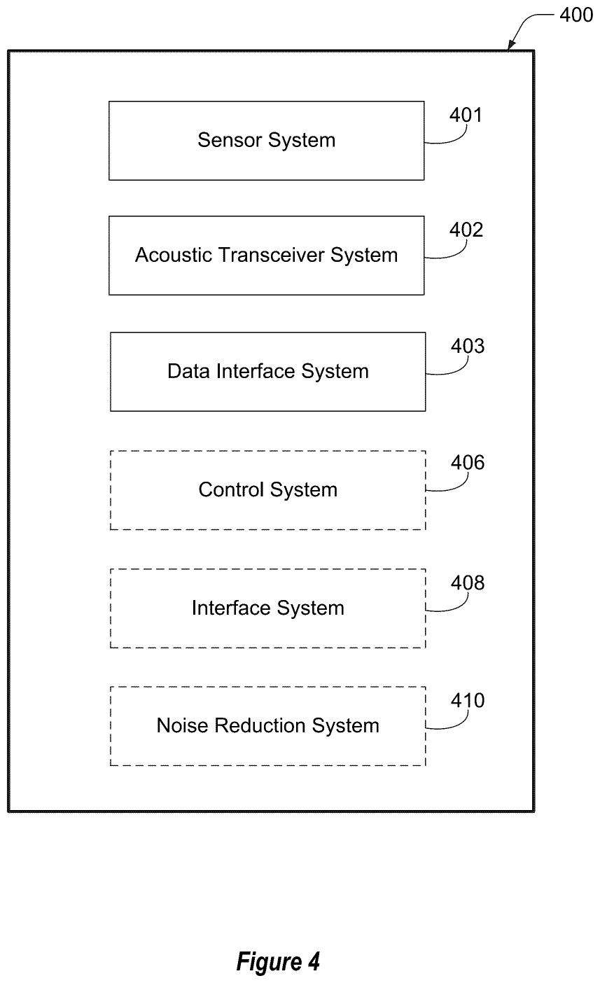

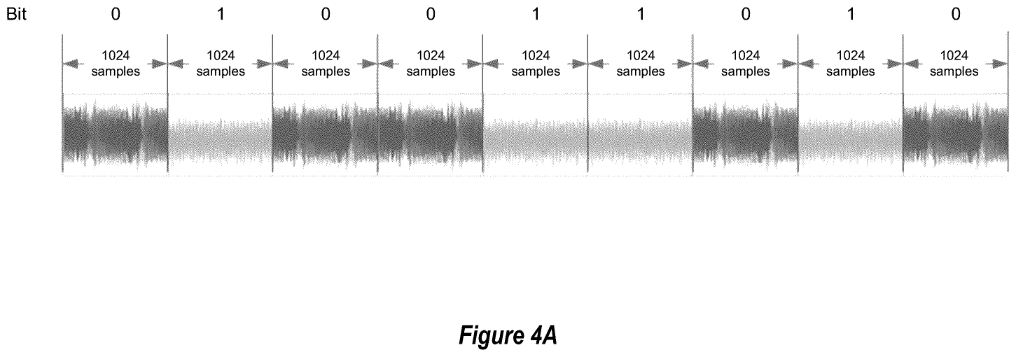

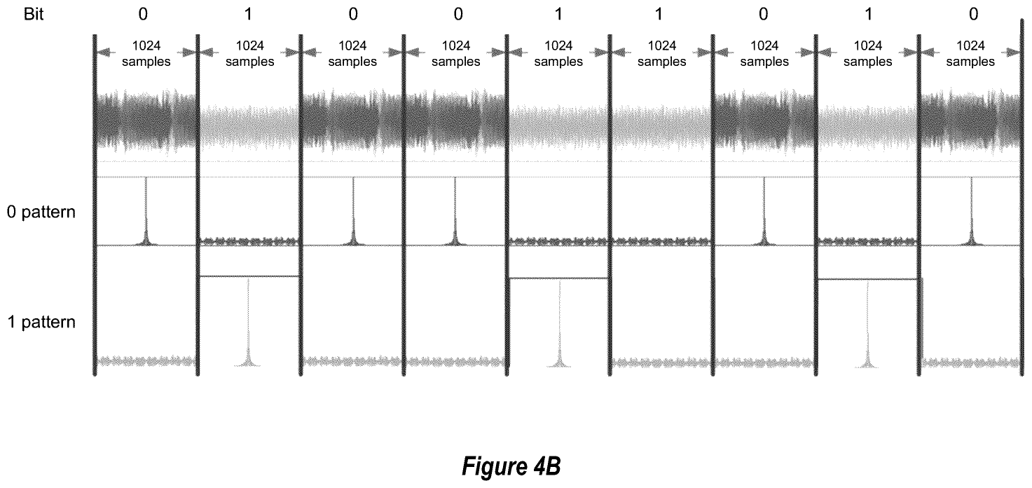

FIG. 4 is block diagram that shows example components of a sensor apparatus 400 according to some implementations. In some embodiments, the sensor apparatus 400 may include a sensor system 401, an acoustic transceiver system 402, and a data interface system 403. Some implementations of the sensor apparatus 400 may include a control system 406, an interface system 408, a noise reduction system 410, or a combination thereof.

In some embodiments, the sensor system 401 may include one or more of various sensors or types of sensors. For example, the sensor system 401 may include at least a photoacoustic sensor system such as the PAPG-based device configured to operate according to the principles described with respect to FIG. 1. In some implementations, the sensor system 401 may be an example of photoacoustic sensor system 300 described above. In another example, the sensor system 401 may include at least an optical sensor system such as a PPG-based device configured to operate according to the principles described with respect to FIG. 2. In other examples, the sensor system 401 may include at least an acoustic sensor (e.g., contact microphone or piezoelectric microphone configured to detect audio vibrations through contact) and/or a bioimpedance sensor (e.g., electrocardiogram (EKG) electrode(s)) or an array thereof.

In some embodiments, the acoustic transceiver system 402 may include an ultrasound transducer or an ultrasound transceiver, which may include an ultrasound transmitter (e.g., ultrasound speaker) configured to emit ultrasonic waves, and an ultrasound receiver (e.g., microphone) configured to detect received ultrasonic waves such as those generated by an ultrasound transmitter, e.g., from another acoustic transceiver system 402 from another sensor apparatus 400.

In some implementations, the acoustic transceiver system 402 may also include an ultrasound codec configured to encode and decode ultrasound signals in the ultrasonic waves. The ultrasound codec may include a digital-to-analog converter (DAC). A DAC-based ultrasound transmitter may generate and cause emission of coded signals, which may allow sending and receiving of data. For example, each bit can be encoded with 1024 samples transmitted at or at about 192 kHz. The bit data can be switched every 1024 samples. Each bit may have the same spectral amplitude between or at about 40 to 70 kHz. However, the phase or waveform of the bits may differ from each another. The phase difference in the bits may allow bits to be distinguished. That is, in an example transmission, a bit value of 0 may be represented by 1024 samples, and a bit value of 1 may be represented by another 1024 samples, as shown in an example encoding by the ultrasound transmitter as illustrated in FIG. 4A.

In some implementations, an ultrasound receiver may be configured to detect whether bit 0 or bit 1 was transmitted based on peak quality and/or amplitude. In some implementations, the start of the ultrasound signals may be detected. Then, each of the 1024 samples in a bit can be evaluated with a 0 pattern and a 1 pattern. The signals may be configured in a way such that cross-correlation of opposite patterns (bit 0 with a 1 pattern and vice versa) will result in low amplitudes, while matching identical patterns will result in high amplitudes, thereby determining which bit the samples correlate to. FIG. 4B illustrates an example decoding by the ultrasound receiver.

Moreover, in some embodiments, ultrasound signals may be orthogonal frequency division multiplexing (OFDM)-modulated so that the ultrasound receiver can demodulate signal from multiple transmission sources.

In some embodiments, ultrasound signals and waves can travel over the air or through tissue. Over-the-air distance can be measured using speed of sound in air and synchronized timing differences among sensor apparatus that have direct line of sight (LOS) between the sensor apparatus or at least between ultrasound transmitter and ultrasound receiver to avoid occlusions or obstructions in the ultrasound transmission path in air. As ultrasound waves are acoustic in nature and thus have mechanical energy, they are particularly sensitive to obstructions blocking their propagation in the air, as compared to, e.g., RF signals. Ultrasound waves over the air may have frequencies between about 22 kHz to about 80 kHz. However, in some embodiments, ultrasound waves may operate in the MHz range for medical equipment (about 2 to 15 MHz).

On the other hand, ultrasound signals transmitted through tissue can be measured using speed of sound in tissue and tissue components (e.g., muscle, blood, water, fat) and synchronized timing differences among sensors. While individual calibration may be performed to determine more exact transmission speeds to account for physiological variations, these ultrasound signals do not suffer from occlusions inside the tissue, as long as there is tight coupling of the sensor with the user's skin. Advantageously, LOS is not required among the sensor apparatus, and distance measurements can be made accurately by ultrasound signals. In certain implementations, a sensor may be an implantable medical device (IMD), which can benefit from exchanging ultrasound signals through tissue.

In some embodiments, ultrasound signal transmission can be switched between over the air to through tissue depending on which path is more advantageous, e.g., if an occlusion is detected in the over-the-air path, or if bone reflections may occur depending on location of sensors. In some implementations, to mitigate any over-the-air blockage, multiple transmit-receive pairs may be used so that not all LOS paths are blocked.

Thus, ultrasound signals can be used to wirelessly transmit information, such as and identifier of the sending sensor and a timestamp associated with a clock timer of the sensing sensor, which are useful for determining a distance between sensors, which will be discussed in further detail below. Separately, RF signaling can be used for wireless communication between sensors, which will now be discussed.

In some embodiments, the data interface system 403 may include one or more transceivers configured to perform wireless data communication (e.g., with another wireless-enabled device) and may be capable of transmitting and receiving radio frequency (RF) signals according to any communication standard, such as any of the Institute of Electrical and Electronics Engineers (IEEE) 802.15.4 standards for ultra-wideband (UWB), IEEE 802.11 standards (including those identified as Wi-Fi® technologies), the Bluetooth® standard, code division multiple access (CDMA), frequency division multiple access (FDMA), time division multiple access (TDMA), Global System for Mobile communications (GSM), GSM/General Packet Radio Service (GPRS), Enhanced Data GSM Environment (EDGE), Terrestrial Trunked Radio (TETRA), Wideband-CDMA (W-CDMA), Evolution Data Optimized (EV-DO), 1×EV-DO, EV-DO Rev A, EV-DO Rev B, High Rate Packet Data (HRPD), High Speed Packet Access (HSPA), High Speed Downlink Packet Access (HSDPA), High Speed Uplink Packet Access (HSUPA), Evolved High Speed Packet Access (HSPA+), Long Term Evolution (LTE), Advanced Mobile Phone System (AMPS), or other known signals that are used to communicate within a wireless, cellular or internet of things (IoT) network, such as a system utilizing 3G, 4G, 5G, 6G, or further implementations thereof.

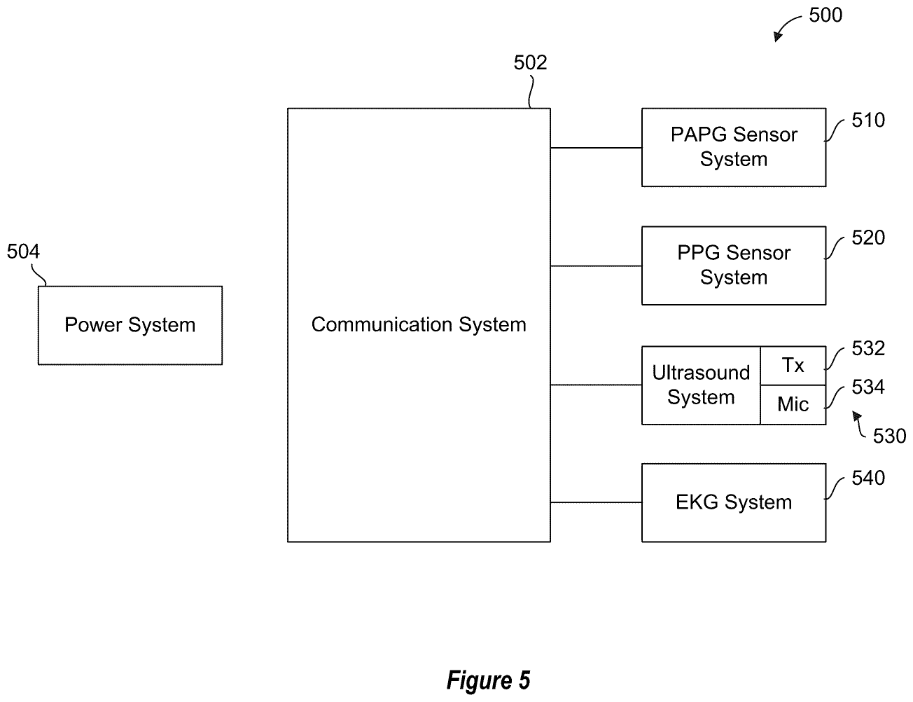

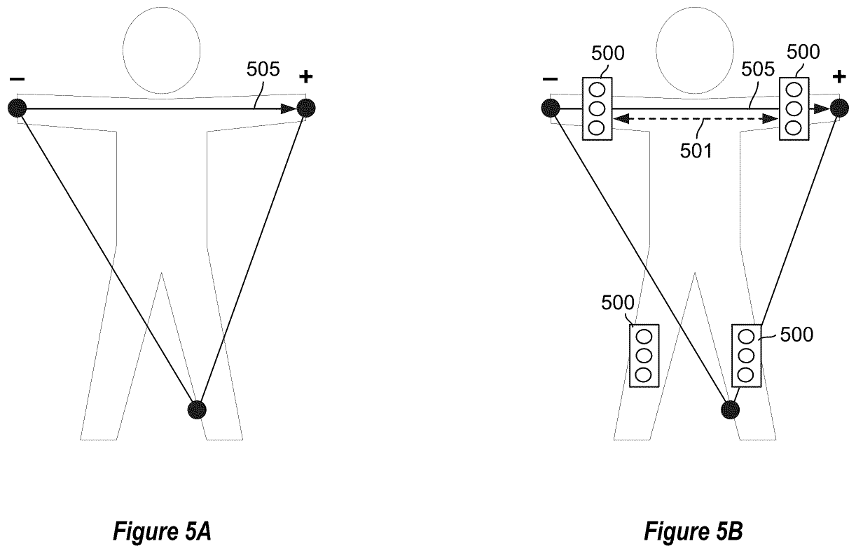

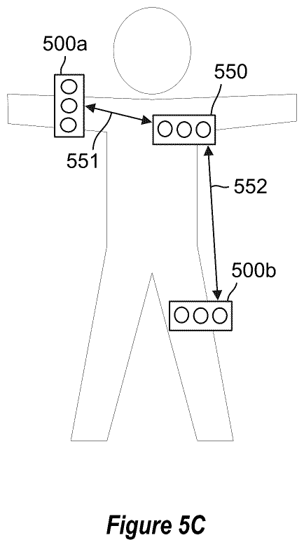

As alluded to by FIG. 5B discussed below, multiple sensor apparatus may communicate with one another over short distances, such as between limb to limb at the most. As such, in some implementations, wireless signals transmitted or received with the data interface system 403 may including RF signals using, for example, Bluetooth (including Bluetooth Low Energy (BLE)). Other communication standards such as those mentioned above could be used in other implementations. In some implementations, the data interface system 403 may additionally or alternatively use non-RF wireless signals, such as infrared signals or other optical technologies.

In some embodiments, the control system 406, the interface system 408, and the noise reduction system 410 may be similar to respective ones of the control system 306, the interface system 308, and the noise reduction system 310 described above and perform similar functionalities for the sensor apparatus 400. In some cases, one or more of the control system 406, the interface system 408, or the noise reduction system 410 may be examples, respectively, of the control system 306, the interface system 308, or the noise reduction system 310. Descriptions for the control system 406, the interface system 408, and the noise reduction system 410 will thus be omitted for sake of brevity. These components, if included with sensor apparatus 400, may be communicatively coupled to other components of the sensor apparatus 400.

FIG. 5 is a block diagram that shows example components of a sensor apparatus 500 according to some implementations. In some embodiments, the sensor apparatus 500 may include a communication system 502, a power system 504, a PAPG sensor system 510, a PPG sensor system 520, an ultrasound system 530 and/or an EKG system 540. Some or all of the PAPG sensor system 510, PPG sensor system 520, ultrasound system 530 and/or EKG system 540, may be examples of the sensors or types of sensors included with sensor system 401.

In some implementations, the communication system 502 may include one or more transceivers configured to perform wireless data communication, e.g., with another wireless-enabled device. The communication system 502 may be an example of the data interface system 403. In some implementations, the communication system 502 may also include a peripheral data interface (e.g., Universal Serial Bus (USB) interface or another serial communication interface) and/or a bus architecture. The communication system 502 may also include memory and/or storage device(s).

In some implementations, the power system 504 may include power interfaces (e.g., power button), a power supply (e.g., battery), a power input port, a power outlet, power management circuitry, or a combination thereof. The power system 504 may thereby be configured to power the sensor apparatus 500 including its components.

In some implementations, the PAPG sensor system 510 may be an example of the photoacoustic sensor system such as the PAPG-based device configured to operate according to the principles described with respect to FIG. 1. In some implementations, the PAPG sensor system 510 may be an example of the photoacoustic sensor system 300 or the sensor system 401. In some implementations, the PPG sensor system 520 may be an example of the optical sensor system such as a PPG-based device configured to operate according to the principles described with respect to FIG. 2. In some implementations, the PPG sensor system 520 may be an example of the sensor system 401. In some implementations, the ultrasound system 530 may be an example of the acoustic transceiver system 402. The ultrasound system 530 may include an ultrasound transmitter 532 and an ultrasound receiver (e.g., microphone) 534.

In some implementations, the EKG system 540 may include one or more leads or electrodes configured to capture EKG waveforms. In 12-lead EKG, 12 separate leads can provide 12 perspectives of the heart's activity from different locations of the body. As is known, such leads may include bipolar limb leads (I, II, III), unipolar limb leads (Augmented Vector Right (AVR) with positive electrode at right shoulder, Augmented Vector Left (AVL) with positive electrode at left shoulder, Augmented Vector Foot (AVF) with positive electrode at the foot or the umbilicus), and six unipolar chest leads (V₁ through V₆). Bipolar leads create a potential between one limb and another limb (− and +). Unipolar leads only require a positive electrode for monitoring. The combination of leads I, II, III, AVR, AVL and AVF creates a hexaxial reference system with each axis 30 degrees apart from one another.

FIG. 5A illustrates an example of a potential 505 occurring via lead I, which can enable electrical differences between a left arm electrode and a right arm electrode to be determined. In certain implementations, the EKG system 540 of the sensor apparatus 500 can include both electrodes, such that, for example, contacting a first electrode of the sensor apparatus 500 with the right limb (e.g., right fingers) can create a potential between the first electrode and a second electrode of the sensor apparatus 500 configured to contact the left limb (e.g., left wrist). As those having ordinary skill in the relevant arts will recognize, lead II involves electrical differences between a left leg electrode and a right arm electrode, and lead III involves electrical differences between a left leg electrode and a left arm electrode.

FIG. 5B illustrates an example of possible positions of a sensor apparatus 500 on a user's limbs at the left arm, the right arm, the left leg, and the right leg. Since the sensor apparatus 500 may include communication system 502, each of the sensor apparatus located on the user's body can exchange information wirelessly with one another, e.g., via communication link 501. Such information may include, e.g., measurements taken by the PAPG sensor system 510, PPG sensor system 520, and/or the EKG system 540, or ultrasound signals sent or received by the ultrasound system 530 with another sensor apparatus. Hence, in some implementations, sensor apparatus 500 on some or all four limbs can obtain EKG signals and synchronize wirelessly. In addition, in some implementations, having the sensor apparatus 500 on some or all four limbs can obtain PWV using other sensing modalities.

FIG. 5C illustrates another example of possible positions of sensor apparatus 500a and 500b on a user's limbs. In some implementations, a relay sensor 550 may be disposed substantially between the sensor apparatus 500a and 500b, which may be useful when a pulse travels in opposite directions toward the sensor apparatus 500a and 500b. In some implementations, each of the sensor apparatus 500a and 500b may be an example of the sensor apparatus 500 and include PAPG sensor system 510, PPG sensor system 520, and ultrasound system 530. In some implementations, the relay sensor 550 may also be an example of the sensor apparatus 500. In some implementations, the relay sensor 550 may have relatively limited functionality, e.g., receive and transmit RF signals for data transmission and time synchronization, and receive and transmit ultrasound signals for distance calculation, and/or determine EKG waveforms for PTT determination, without performing photoacoustic or optical sensing. Said time synchronization and distance calculation will be discussed in greater detail elsewhere herein, e.g., with respect to FIGS. 11-14B.

The opposite directions mentioned above may be left wrist and right wrist, neck aortic and femoral, etc. In such configurations, the PWV may be based on the difference of the sensor distances rather than the sum of the sensor distances. Hence, in the FIG. 5C configuration, PWV may be determined by way of the following relationship between distances between the sensor apparatus 500a and 500b and the relay sensor 550:

$$PWV = \vert {d}_{2 r} - {d}_{1 r} \vert / PTT \qquad ( Eqn . 1 )$$

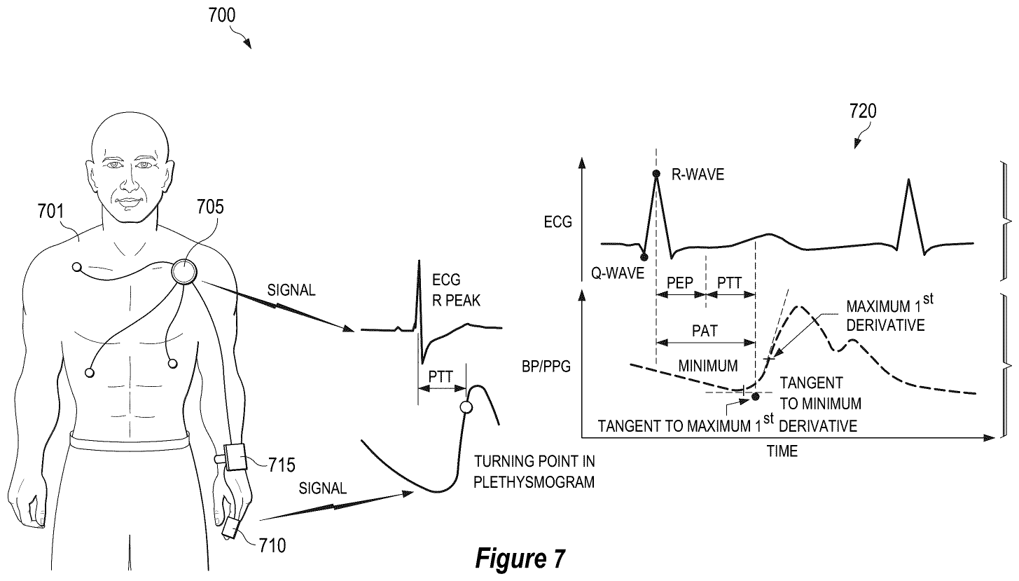

Here, d₁ᵣ is a distance 551 between a first sensor (e.g., sensor apparatus 500a) and the relay sensor 550, and d₂ᵣ is a distance 552 between a second sensor (e.g., sensor apparatus 500b) and the relay sensor 550. PTT is a pulse transmit time between the first sensor and the second sensor, which can be measured using EKG as shown in FIG. 7.

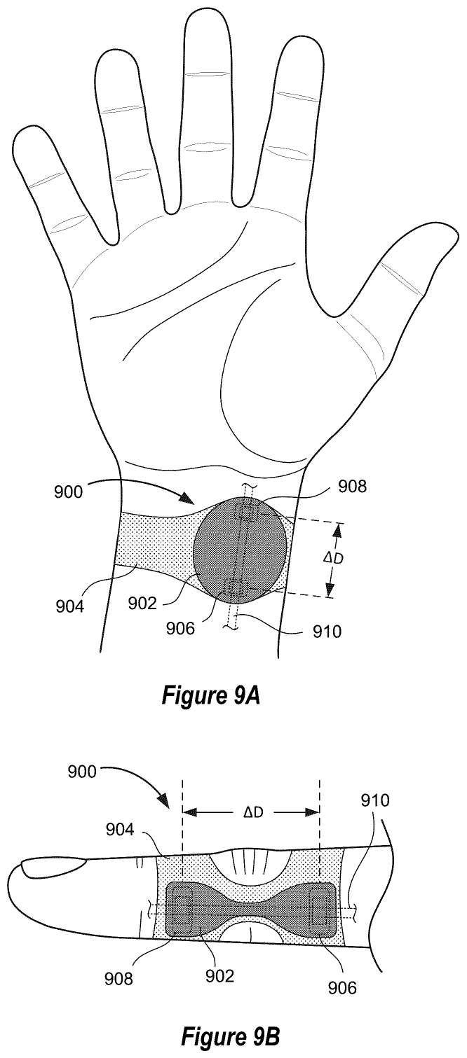

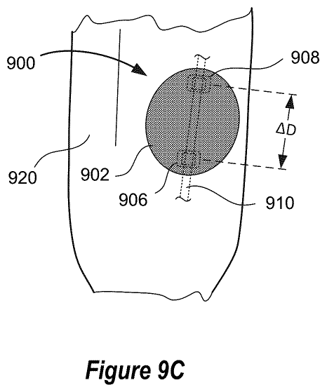

In some embodiments, the sensor apparatus 400 or the sensor apparatus 500 may be implemented as a wearable structure such as a wearable housing having a small chassis. One or more above components may be disposed within the wearable structure. These components, including the sensors, can be low power, low cost, and very compact, and possess high sensitivity and an ability to be mass produced to be integrated in various consumer devices, including wearable devices. Any such component may abut a surface of the wearable structure such that the component is capable of making contact with a user's skin. For example, the interface and/or contact portion of the sensor system 401 may be configured to make contact with the skin. The wearable device may, for example, be a bracelet, an armband, a wristband, a watch, a ring, a headband or a patch. In certain embodiments, the wearable device may be one or a pair of earbuds, a headset, a head rest mount, a headband, headphones, or another head-wearable or head-mounted device. Illustrative examples of these implementations can be seen in FIGS. 9A-9C, but the sensor apparatus 400 or the sensor apparatus 500 can be in any form of wearable device, including those mentioned herein.

Advantageously, the sensor apparatus being wearable may be useful in conjunction with performing wireless data communication with the communication system 502. Moreover, the wearability of devices having a small chassis increases the flexibility in the placement of the sensor apparatus since the sensor apparatus can be placed nearly anywhere on the body. This can simplify the setup procedure and usability of the sensors.

In some embodiments, the sensor apparatus 500 may further include other types of sensors (not shown), such as a temperature sensor, a force sensor, a capacitance sensor, an inertial sensor (e.g., gyroscope, accelerometer), or a combination thereof.

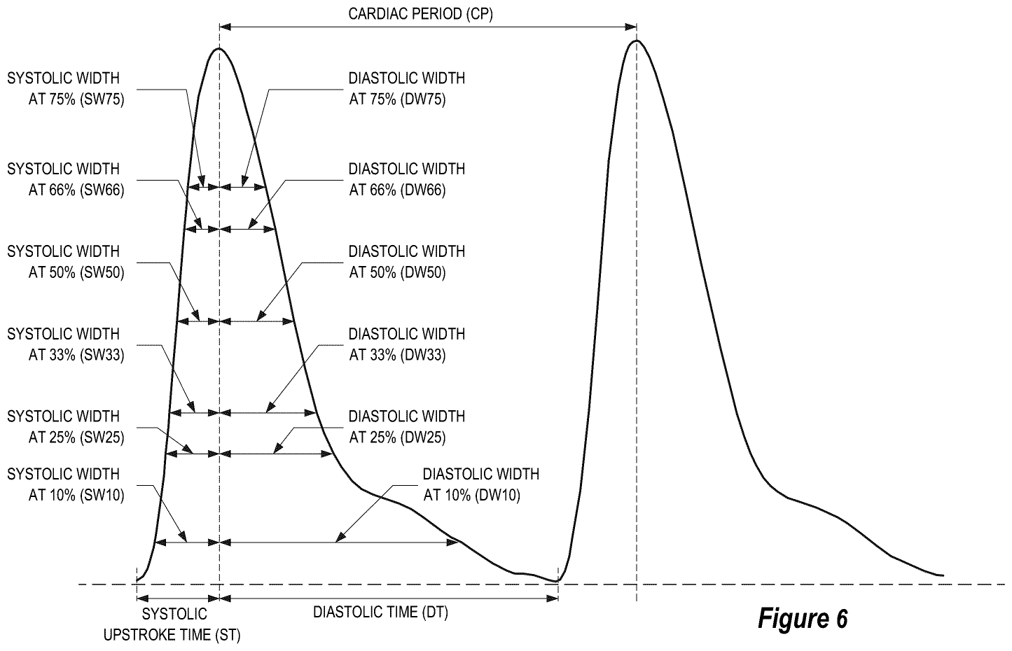

FIG. 6 shows examples of heart rate waveform (HRW) features that may be extracted according to some implementations. The horizontal axis of FIG. 6 represents time and the vertical axis represents signal amplitude. The cardiac period is indicated by the time between adjacent peaks of the HRW. The systolic and diastolic time intervals are indicated below the horizontal axis. During the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

The HRW features that are illustrated in FIG. 6 pertain to the width of the systolic and/or diastolic portions of the HRW curve at various “heights,” which are indicated by a percentage of the maximum amplitude. For example, the SW50 feature is the width of the systolic portion of the HRW curve at a “height” of 50% of the maximum amplitude. In some implementations, the HRW features used for blood pressure estimation may include some or all of the SW10, SW25, SW33, SW50, SW66, SW75, DW10, DW25, DW33, DW50, DW66 and DW75 HRW features. In other implementations, additional HRW features may be used for blood pressure estimation. Such additional HRW features may, in some instances, include the sum and ratio of the SW and DW at one or more “heights,” e.g., (DW75+SW75), DW75/SW75, (DW66+SW66), DW66/SW66, (DW50+SW50), DW50/SW50, (DW33+SW33), DW33/SW33, (DW25+SW25), DW25/SW25 and/or (DW10+SW10), DW10/SW10. Other implementations may use yet other HRW features for blood pressure estimation. Such additional HRW features may, in some instances, include sums, differences, ratios and/or other operations based on more than one “height,” such as (DW75+SW75)/(DW50+SW50), (DW50+SW50/(DW10+SW10), etc.

FIG. 7 shows examples of devices that may be used in a system for estimating blood pressure based, at least in part, on pulse transit time (PTT). As with other figures provided herein, the numbers, types and arrangements of elements are merely presented by way of example. According to this example, the system 700 includes at least two sensors. In this example, the system 700 includes at least an electrocardiogram sensor 705 and a device 710 that is configured to be mounted on a finger of the person 701. In this example, the device 710 is, or includes, an apparatus configured to perform at least some PAPG methods disclosed herein. For example, the device 710 may be, or may include, the sensor apparatus 400 of FIG. 4 or a similar apparatus.

As noted in the graph 720, the PAT includes two components, the pre-ejection period (PEP), the time needed to convert the electrical signal into a mechanical pumping force and isovolumetric contraction to open the aortic valves) and the PTT. The starting time for the PAT can be estimated based on the QRS complex—an electrical signal characteristic of the electrical stimulation of the heart ventricles. As shown by the graph 720, in this example the beginning of a pulse arrival time (PAT) may be calculated according to an R-Wave peak measured by the electrocardiogram sensor 705 and the end of the PAT may be detected via analysis of signals provided by the device 710. In this example, the end of the PAT is assumed to correspond with an intersection between a tangent to a local minimum value detected by the device 710 and a tangent to a maximum slope/first derivative of the sensor signals after the time of the minimum value.

There are many known algorithms for blood pressure estimation based on the PTT and/or the PAT, some of which are summarized in Table 1 and described in the corresponding text on pages 5-10 of Sharma, M. et al., Cuff-Less and Continuous Blood Pressure Monitoring: a Methodological Review (“Sharma”), in Multidisciplinary Digital Publishing Institute (MDPI) Technologies 2017, 5, 21, both of which are hereby incorporated by reference.

Some previously disclosed methods have involved calculating blood pressure according to one or more of the equations shown in Table 1 of Sharma, or other known equations, based on a PTT and/or PAT measured by a sensor system that includes a PPG sensor. As noted above, some disclosed PAPG-based implementations are configured to distinguish artery HRWs from other HRWs. Such implementations may provide more accurate measurements of the PTT and/or PAT, relative to those measured by a PPG sensor. Therefore, disclosed PAPG-based implementations may provide more accurate blood pressure estimations, even when the blood pressure estimations are based on previously-known formulae.

Other implementations of the system 700 may not include the electrocardiogram sensor 705. In some such implementations, the device 715, which is configured to be mounted on a wrist of the person 701, may be, or may include, an apparatus configured to perform at least some PAPG methods disclosed herein. For example, the device 715 may be, or may include, the sensor apparatus 400 of FIG. 4 or a similar apparatus. According to some such examples, the device 715 may include a light source system and two or more ultrasonic receivers. One example is described below with reference to FIG. 9A. In some examples, the device 715 may include an array of ultrasonic receivers.

In some implementations of the system 700 that do not include the electrocardiogram sensor 705, the device 710 may include a light source system and two or more ultrasonic receivers. One example is described below with reference to FIG. 9B.

FIG. 8 shows a cross-sectional side view of a diagrammatic representation of a portion of an artery 800 through which a pulse 802 is propagating. The block arrow in FIG. 8 shows the direction of blood flow and pulse propagation. As diagrammatically shown, the propagating pulse 802 causes strain in the arterial walls 804, which is manifested in the form of an enlargement in the diameter (and consequently the cross-sectional area) of the arterial walls—referred to as “distension.” The spatial length L of an actual propagating pulse along an artery (along the direction of blood flow) is typically comparable to the length of a limb, such as the distance from a subject's shoulder to the subject's wrist or finger, and is generally less than one meter (m). However, the length L of a propagating pulse can vary considerably from subject to subject, and for a given subject, can vary significantly over durations of time depending on various factors. The spatial length L of a pulse will generally decrease with increasing distance from the heart until the pulse reaches capillaries.

As described above, some particular implementations relate to devices, systems and methods for estimating blood pressure or other cardiovascular characteristics based on estimates of an arterial distension waveform. The terms “estimating,” “measuring,” “calculating,” “inferring,” “deducing,” “evaluating,” “determining” and “monitoring” may be used interchangeably herein where appropriate unless otherwise indicated. Similarly, derivations from the roots of these terms also are used interchangeably where appropriate; for example, the terms “estimate,” “measurement,” “calculation,” “inference” and “determination” also are used interchangeably herein. In some implementations, the pulse wave velocity (PWV) of a propagating pulse may be estimated by measuring the pulse transit time (PTT) of the pulse as it propagates from a first physical location along an artery to another more distal second physical location along the artery. However, either version of the PTT may be used for the purpose of blood pressure estimation. Assuming that the physical distance ΔD between the first and the second physical locations is ascertainable, the PWV can be estimated as the quotient of the physical spatial distance ΔD traveled by the pulse divided by the time (PTT) the pulse takes in traversing the physical spatial distance ΔD. Generally, a first sensor positioned at the first physical location is used to determine a starting time (also referred to herein as a “first temporal location”) at which point the pulse arrives at or propagates through the first physical location. A second sensor at the second physical location is used to determine an ending time (also referred to herein as a “second temporal location”) at which point the pulse arrives at or propagates through the second physical location and continues through the remainder of the arterial branch. In such examples, the PTT represents the temporal distance (or time difference) between the first and the second temporal locations (the starting and the ending times).

The fact that measurements of the arterial distension waveform are performed at two different physical locations implies that the estimated PWV inevitably represents an average over the entire path distance ΔD through which the pulse propagates between the first physical location and the second physical location. More specifically, the PWV generally depends on a number of factors including the density of the blood p, the stiffness E of the arterial wall (or inversely the elasticity), the arterial diameter, the thickness of the arterial wall, and the blood pressure. Because both the arterial wall elasticity and baseline resting diameter (for example, the diameter at the end of the ventricular diastole period) vary significantly throughout the arterial system, PWV estimates obtained from PTT measurements are inherently average values (averaged over the entire path length ΔD between the two locations where the measurements are performed).

In traditional methods for obtaining PWV, the starting time of the pulse has been obtained at the heart using an electrocardiogram (EKG) sensor, which detects electrical signals from the heart. For example, the starting time can be estimated based on the QRS complex—an electrical signal characteristic of the electrical stimulation of the heart ventricles. In such approaches, the ending time of the pulse is typically obtained using a different sensor positioned at a second location (for example, a finger). As a person having ordinary skill in the art will appreciate, there are numerous arterial discontinuities, branches, and variations along the entire path length from the heart to the finger. The PWV can change by as much as or more than an order of magnitude along various stretches of the entire path length from the heart to the finger. As such, traditional PWV estimates based on such long path lengths are unreliable.

In various implementations described herein, PTT estimates are obtained based on measurements (also referred to as “arterial distension data” or more generally as “sensor data”) associated with an arterial distension signal obtained by each of a first arterial distension sensor 806 and a second arterial distension sensor 808 proximate first and second physical locations, respectively, along an artery of interest. In some particular implementations, the first arterial distension sensor 806 and the second arterial distension sensor 808 are advantageously positioned proximate first and second physical locations between which arterial properties of the artery of interest, such as wall elasticity and diameter, can be considered or assumed to be relatively constant. In this way, the PWV calculated based on the PTT estimate is more representative of the actual PWV along the particular segment of the artery. In turn, the blood pressure P estimated based on the PWV is more representative of the true blood pressure. In some implementations, the magnitude of the distance ΔD of separation between the first arterial distension sensor 806 and the second arterial distension sensor 808 (and consequently the distance between the first and the second locations along the artery) can be in the range of about 1 centimeter (cm) to tens of centimeters-long enough to distinguish the arrival of the pulse at the first physical location from the arrival of the pulse at the second physical location, but close enough to provide sufficient assurance of arterial consistency. In some specific implementations, the distance ΔD between the first and the second arterial distension sensors 806 and 808 can be in the range of about 1 cm to about 30 cm, and in some implementations, less than or equal to about 20 cm, and in some implementations, less than or equal to about 10 cm, and in some specific implementations less than or equal to about 5 cm. In some other implementations, the distance ΔD between the first and the second arterial distension sensors 806 and 808 can be less than or equal to 1 cm, for example, about 0.1 cm, about 0.25 cm, about 0.5 cm or about 0.75 cm. By way of reference, a typical PWV can be about 15 meters per second (m/s). Using a monitoring device in which the first and the second arterial distension sensors 806 and 808 are separated by a distance of about 5 cm, and assuming a PWV of about 15 m/s implies a PTT of approximately 3.3 milliseconds (ms).

The value of the magnitude of the distance ΔD between the first and the second arterial distension sensors 806 and 808, respectively, can be preprogrammed into a memory within a monitoring device that incorporates the sensors (for example, such as a memory of, or a memory configured for communication with a control system such as 406). As will be appreciated by a person of ordinary skill in the art, the spatial length L of a pulse can be greater than the distance ΔD from the first arterial distension sensor 806 to the second arterial distension sensor 808 in such implementations. As such, although the diagrammatic pulse 802 shown in FIG. 8 is shown as having a spatial length L comparable to the distance between the first arterial distension sensor 806 and the second arterial distension sensor 808, in actuality each pulse can typically have a spatial length L that is greater and even much greater than (for example, about an order of magnitude or more than) the distance ΔD between the first and the second arterial distension sensors 806 and 808.

Implementations described herein using multiple distinct sensors of different modalities (e.g., PAPG and PPG) can enable more flexible placement of sensors compared to a single-modality approach, and thus flexible configurations for measurement of global PWV, which as noted above was traditionally difficult. Global PWV may refer to PWV measured across two distinct blood vessels over a larger arterial trajectory, whereas local PWV may refer to PWV measured from a single arterial site or locally across piecewise segments of individual target arteries, such as that shown in FIG. 8. Single-modality measurements (e.g., PPG and another PPG, PAPG and another PAPG) are more typically used for local PWV, which is also possible using the disclosed implementations.

In some implementations of the monitoring devices disclosed herein, both the first arterial distension sensor 806 and the second arterial distension sensor 808 are sensors of the same sensor type. In some such implementations, the first arterial distension sensor 806 and the second arterial distension sensor 808 are identical sensors. In such implementations, each of the first arterial distension sensor 806 and the second arterial distension sensor 808 utilizes the same sensor technology with the same sensitivity to the arterial distension signal caused by the propagating pulses, and has the same time delays and sampling characteristics. In some implementations, each of the first arterial distension sensor 806 and the second arterial distension sensor 808 is configured for photoacoustic plethysmography (PAPG) sensing, e.g., as disclosed elsewhere herein. Some such implementations include a light source system and two or more ultrasonic receivers. In some implementations, each of the first arterial distension sensor 806 and the second arterial distension sensor 808 is configured for ultrasound sensing via the transmission of ultrasonic signals and the receipt of corresponding reflections. In some alternative implementations, each of the first arterial distension sensor 806 and the second arterial distension sensor 808 may be configured for impedance plethysmography (IPG) sensing, also referred to in biomedical contexts as bioimpedance sensing. In various implementations, whatever types of sensors are utilized, each of the first and the second arterial distension sensors 806 and 808 broadly functions to capture and provide arterial distension data indicative of an arterial distension signal resulting from the propagation of pulses through a portion of the artery proximate to which the respective sensor is positioned. For example, the arterial distension data can be provided from the sensor to a processor in the form of voltage signal generated or received by the sensor based on an ultrasonic signal or an impedance signal sensed by the respective sensor.

As described above, during the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

In the context of bioimpedance sensing (or impedance plethysmography), the blood in the arteries has a greater electrical conductivity than that of the surrounding or adjacent skin, muscle, fat, tendons, ligaments, bone, lymph or other tissues. The susceptance (and thus the permittivity) of blood also is different from the susceptances (and permittivities) of the other types of surrounding or nearby tissues. As a pulse propagates through a particular location, the corresponding increase in the volume of blood results in an increase in the electrical conductivity at the particular location (and more generally an increase in the admittance, or equivalently a decrease in the impedance). Conversely, during the diastolic phase of the cardiac cycle, the corresponding decrease in the volume of blood results in an increase in the electrical resistivity at the particular location (and more generally an increase in the impedance, or equivalently a decrease in the admittance).

A bioimpedance sensor generally functions by applying an electrical excitation signal at an excitation carrier frequency to a region of interest via two or more input electrodes, and detecting an output signal (or output signals) via two or more output electrodes. In some more specific implementations, the electrical excitation signal is an electrical current signal injected into the region of interest via the input electrodes. In some such implementations, the output signal is a voltage signal representative of an electrical voltage response of the tissues in the region of interest to the applied excitation signal. The detected voltage response signal is influenced by the different, and in some instances time-varying, electrical properties of the various tissues through which the injected excitation current signal is passed. In some implementations in which the bioimpedance sensor is operable to monitor blood pressure, heartrate or other cardiovascular characteristics, the detected voltage response signal is amplitude- and phase-modulated by the time-varying impedance (or inversely the admittance) of the underlying arteries, which fluctuates synchronously with the user's heartbeat as described above. To determine various biological characteristics, information in the detected voltage response signal is generally demodulated from the excitation carrier frequency component using various analog or digital signal processing circuits, which can include both passive and active components.

In some examples incorporating ultrasound sensors, measurements of arterial distension may involve directing ultrasonic waves into a limb towards an artery, for example, via one or more ultrasound transducers. Such ultrasound sensors also are configured to receive reflected waves that are based, at least in part, on the directed waves. The reflected waves may include scattered waves, specularly reflected waves, or both scattered waves and specularly reflected waves. The reflected waves provide information about the arterial walls, and thus the arterial distension.

In some implementations, regardless of the type of sensors utilized for the first arterial distension sensor 806 and the second arterial distension sensor 808, both the first arterial distension sensor 806 and the second arterial distension sensor 808 can be arranged, assembled or otherwise included within a single housing of a single monitoring device. As described above, the housing and other components of the monitoring device can be configured such that when the monitoring device is affixed or otherwise physically coupled to a subject, both the first arterial distension sensor 806 and the second arterial distension sensor 808 are in contact with or in close proximity to the skin of the user at first and second locations, respectively, separated by a distance ΔD, and in some implementations, along a stretch of the artery between which various arterial properties can be assumed to be relatively constant. In various implementations, the housing of the monitoring device is a wearable housing or is incorporated into or integrated with a wearable housing. In some specific implementations, the wearable housing includes (or is connected with) a physical coupling mechanism for removable non-invasive attachment to the user. The housing can be formed using any of a variety of suitable manufacturing processes, including injection molding and vacuum forming, among others. In addition, the housing can be made from any of a variety of suitable materials, including, but not limited to, plastic, metal, glass, rubber and ceramic, or combinations of these or other materials. In particular implementations, the housing and coupling mechanism enable full ambulatory use. In other words, some implementations of the wearable monitoring devices described herein are noninvasive, not physically-inhibiting and generally do not restrict the free uninhibited motion of a subject's arms or legs, enabling continuous or periodic monitoring of cardiovascular characteristics such as blood pressure even as the subject is mobile or otherwise engaged in a physical activity. As such, the monitoring device facilitates and enables long-term wearing and monitoring (for example, over days, weeks or a month or more without interruption) of one or more biological characteristics of interest to obtain a better picture of such characteristics over extended durations of time, and generally, a better picture of the user's health.

In some implementations, the monitoring device can be positioned around a wrist of a user with a strap or band, similar to a watch or fitness/activity tracker. FIG. 9A shows an example monitoring device 900 designed to be worn around a wrist according to some implementations. In the illustrated example, the monitoring device 900 includes a housing 902 integrally formed with, coupled with or otherwise integrated with a wristband 904. The first and the second arterial distension sensors 906 and 908 may, in some instances, each include an instance of the ultrasonic receiver system and a portion of the light source system that are described above. In this example, the monitoring device 900 is coupled around the wrist such that the first and the second arterial distension sensors 906 and 908 within the housing 902 are each positioned along a segment of the radial artery 910 (note that the sensors are generally hidden from view from the external or outer surface of the housing facing the subject while the monitoring device is coupled with the subject, but exposed on an inner surface of the housing to enable the sensors to obtain measurements through the subject's skin from the underlying artery). Also as shown, the first and the second arterial distension sensors 906 and 908 are separated by a fixed distance ΔD. In some other implementations, the monitoring device 900 can similarly be designed or adapted for positioning around a forearm, an upper arm, an ankle, a lower leg, an upper leg, or a finger (all of which are hereinafter referred to as “limbs”) using a strap or band.

FIG. 9B shows an example monitoring device 900 designed to be worn on a finger according to some implementations. The first and the second arterial distension sensors 906 and 908 may, in some instances, each include an instance of the ultrasonic receiver and a portion of the light source system that are described above.

In some other implementations, the monitoring devices disclosed herein can be positioned on a region of interest of the user without the use of a strap or band. For example, the first and the second arterial distension sensors 906 and 908 and other components of the monitoring device can be enclosed in a housing that is secured to the skin of a region of interest of the user using an adhesive or other suitable attachment mechanism (an example of a “patch” monitoring device).

FIG. 9C shows an example monitoring device 900 designed to reside on an earbud according to some implementations. According to this example, the monitoring device 900 is coupled to the housing of an earbud 920. The first and second arterial distension sensors 906 and 908 may, in some instances, each include an instance of the ultrasonic receiver and a portion of the light source system that are described above.

### Example Sensor Configurations

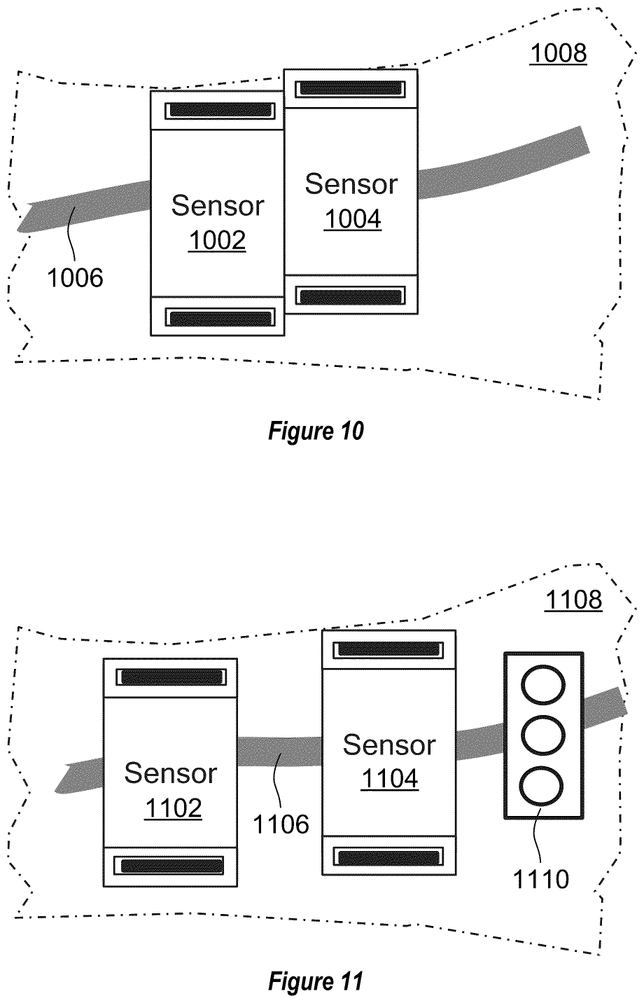

FIG. 10 is a diagram showing an example of sensor apparatus 1002, 1004 coupled physically. In this example, the sensor apparatus 1002, 1004 may be disposed over a target object, such as a blood vessel 1006 inside tissue 1008 of a user. The sensor apparatus 1002, 1004 may communicate (e.g., sensor measurement data) via wired connection. Depending on the variation, there may be a physical communication link (e.g., a cable), or the sensor apparatus 1002, 1004 may be in contact with each other, or both. Hence, the sensor apparatus 1002, 1004 are local sensors separated by a short distance on the order of the size of the sensor apparatus, such as a few centimeters, and capable of reliably measuring local PWV only. Sensors connected by wire may be bulky and not flexible to deploy. However, as mentioned elsewhere herein, the present disclosure advantageously enables small, compact devices to determine physiological characteristics and parameters while being flexible in placement around the body, allowing reliable global PWV measurements as well.

FIG. 11 is a diagram showing an example of sensor apparatus 1102, 1104 coupled and synchronized wirelessly. Each of sensor apparatus 1102, 1104 may an example of sensor apparatus 400 or sensor apparatus 500, and thus may include at least a sensor (e.g., sensor system 401, PAPG sensor system 510, PPG sensor system 520, EKG system 540, or a combination thereof), an acoustic transceiver (e.g., acoustic transceiver system 402 or ultrasound system 530), and other (e.g., RF) communication means (e.g., data interface system 403, communication system 502). More pointedly, sensor apparatus 1102, 1104 may be configured to communicate with each other as well as other wireless-enabled devices. One example of such a wireless-enabled device is a host device (e.g., a smartphone), which will be discussed further below and become more relevant to achieving the synchronized physiological measurements with the multiple sensors according to the present disclosure.

Referring back to the example of FIG. 11, the sensor apparatus 1102, 1104 may be disposed over a target object, such as a blood vessel 1106 inside tissue 1108 of the user, similar to the example of FIG. 10. Contrast with the sensor apparatus 1002, 1004 of FIG. 10, which are coupled and connected physically, and may even be in contact with each other. Unlike the example of FIG. 10, the blood vessel 1106 may extend within one limb, or extend from one limb to another. That is, the blood vessel 1106 and the positions of the sensor apparatus 1102, 1104 are not shown to scale. The sensor apparatus 1102, 1104 may, depending on implementation, be separated by a distance that is orders of magnitude larger than that between the sensor apparatus 1002, 1004 (e.g., a few meters).

Since the sensor apparatus 1102, 1104 are not in physically coupled with each other, they may be linked wirelessly via RF signaling and communication, e.g., using a Bluetooth connection or via another type of short-range RF signaling mentioned elsewhere herein (e.g., with respect to data interface system 403). Moreover, the sensor apparatus 1102, 1104 may communicate acoustically, e.g., using ultrasound signals as discussed above with respect to the acoustic transceiver system 402 and encoding and decoding schemes in FIGS. 4A and 4B. In some implementations, an acoustics-only sensor such as sensor 1110 may only have an acoustic transceiver system (e.g., microphone or microphone array) and may be used for acoustic communication with the sensor apparatus 1102, 1104 or other sensors.

Thus, wireless synchronization of multiple sensor apparatus and accurate estimation of physiological characteristics (e.g., PTT, PWV) across the body and physiological parameter of the user (e.g., blood pressure) is possible. More specifically, a given sensor apparatus can be configured to acquire measurement data, wireless communication can allow the sensor apparatus to transmit measurement data and clock data to (or receive from) another sensor apparatus, and acoustic (e.g., ultrasound) signals can be used to calculate or determine an accurate distance between multiple sensor apparatus. In some implementations, acoustic signals can also be used to transmit or receive data (e.g., clock timestamps, metadata regarding sensor apparatus) using encoding and decoding schemes discussed above. While ultrasound signaling can have a smaller distance resolution than RF signaling and thus have high accuracy for measuring distance, RF signaling can be used in certain implementations to determine distances between sensor apparatus, e.g., if sensor apparatus is placed far apart on the body, e.g., on opposite arm and leg.

Advantageously, using the wireless synchronization enables reduction of individual sensor size and measurement of local characteristics (e.g., PWV) from different body locations simultaneously. The latter may assist with improving prediction of parameters such as blood pressure of the user, including by improving a machine learning model used in conjunction with determination of the blood pressure.

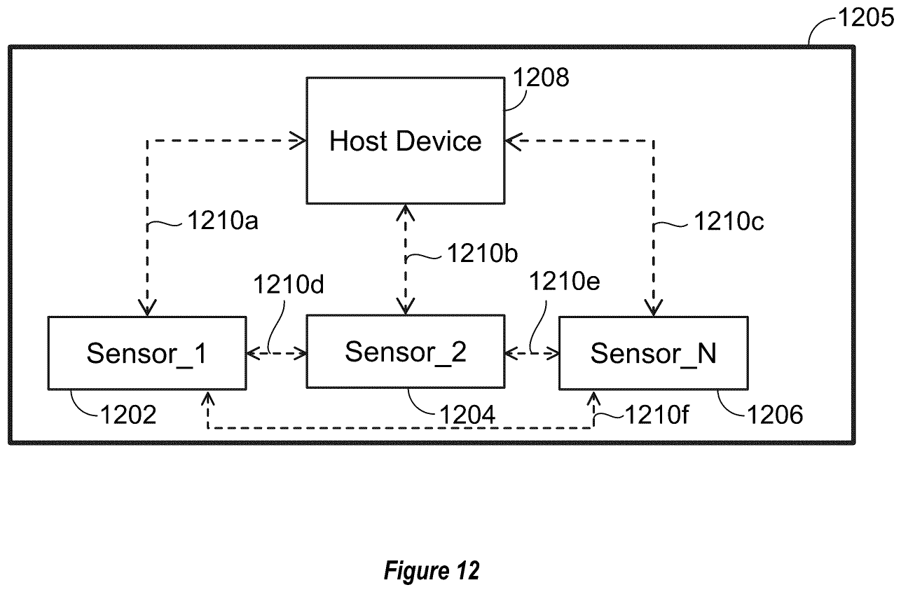

FIG. 12 is a block diagram that shows an example configuration of sensors 1202, 1204, 1206 wirelessly synchronized with a host device 1208 according to some implementations. In some embodiments, each of the sensors 1202, 1204, 1206 (labeled Sensor_1, Sensor_2, and Sensor_N, respectively) and may be an example of sensor apparatus 400, 500, 1102 or 1104 and thus may be capable of wireless communication with other sensors and the host device 1208. Such wireless communications may be conducted via wireless communication links 1210a-1210f among sensors 1202, 1204, 1206 and between various ones of the sensors 1202, 1204, 1206 and the host device 1208. In some implementations, the wireless communication links 1210a-1210f may each be an RF communication link established via, e.g., Bluetooth. Bluetooth is a low-power and power-efficient standard that is effective for short distances. However, as said earlier, other short-range communication standards may be used in other implementations. In some embodiments, the host device 1208 may be a wireless-enabled device, e.g., smartphone, tablet, smartwatch, display, or any other electronic devices listed elsewhere herein. It will be appreciated that any similar wireless and/or networked device capable of transmitting and/or receiving digital data can be the host device 1208. In addition, although three sensors are depicted in FIG. 12, it is understood that there may be two sensors (e.g., two of the three sensors shown) or more than three sensors (e.g., one on each limb and/or additional wearable devices such as a waistband or additional sensors on a limb) in a configuration of sensors. The number of synchronized sensors can be a function of acceptable error threshold and performance. The following discussion will apply with similar effect to sensor setups and configurations having two or more sensors capable of communicating with one another via RF signals and acoustic signals. More specifically, each sensor 1202, 1204, 1206 is a node secured about a user's body, and may have an ultrasound transmitter and an ultrasound receiver in some implementations. Each sensor 1202, 1204, 1206 may be configured to communicate sensor data with the host device 1208 via RF signals or ultrasound signals.

In some embodiments, sensor data may be exchanged in a time-synchronized or time-compensated manner to determine an accurate measurement of physiological characteristics and parameters. High precision can be reached by utilizing timestamping (e.g., medium access control (MAC)-layer timestamp) and error compensation including clock skew estimation via regression. One or more delays may also occur between different protocol stack layers. Each sensor may have a local clock exhibiting the typical timing errors of crystals and can communicate over an error-corrected wireless link to the host device 1208 or neighbor sensor(s). Robust time synchronization can be achieved by utilizing periodic one-way synchronization messages or two-way synchronization messages. The host device 1208 and sensors 1202, 1204, 1206 may exchange their local timestamps using one-way or two-way messages during a transmission time interval.

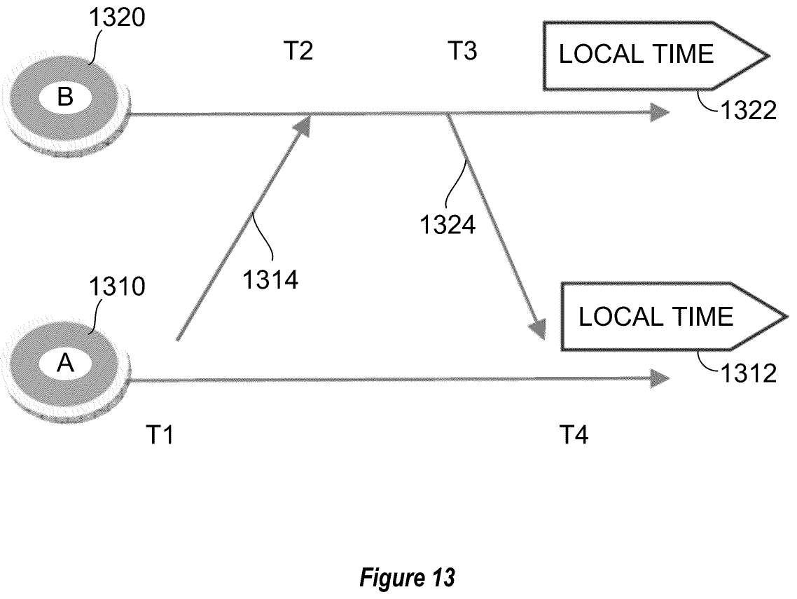

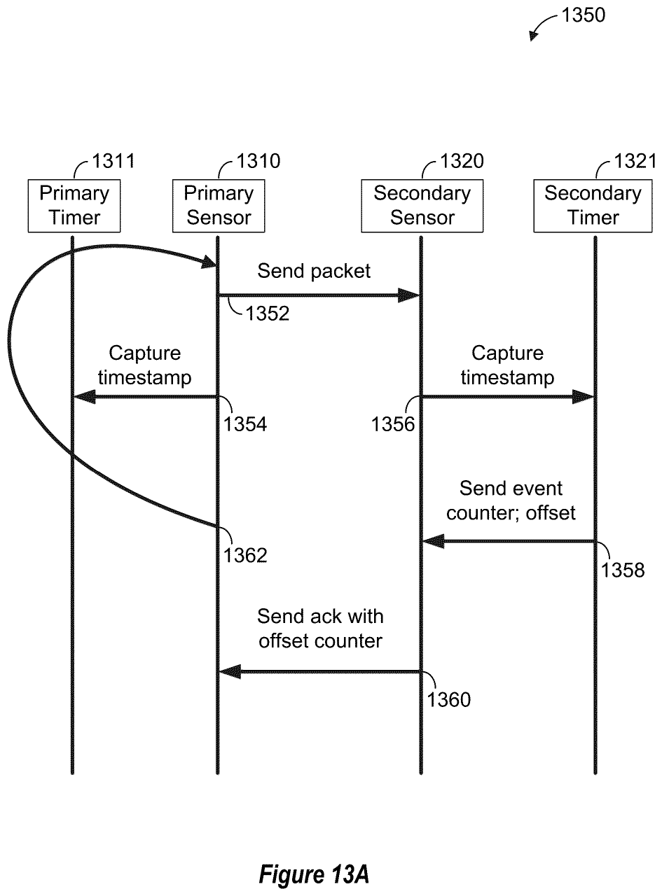

FIG. 13 shows an example time synchronization between sensors 1310, 1320 via timestamp exchange. The time synchronization can occur between any two given sensors. One of the sensors may be considered a primary or central sensor node, and the other one of the sensors may be considered a secondary or peripheral sensor node. For example, a first sensor 1310 may be the primary or central sensor node, and a second sensor 1320 may be the secondary or peripheral sensor node. A timestamp may be generated according to each sensor's respective local clock times 1312, 1322. The clocks of the sensors 1310, 1320 may run on their own clocks independently but can use RF signaling of timestamp data to adjust their local clock timers according to a synchronization procedure. In some implementations, position information can additionally be encoded in ultrasound signals to help identify a need to use a relay sensor (e.g., relay sensor 510 of FIG. 5C). In one example approach, the second sensor 1320 may adjust its local clock timer by using the timestamp of the first sensor 1310 at T1 and known parameters. More specifically, the following relations among parameters and timestamp may be used:

$$T {S}_{P} [ m ] = {β}_{0} + {β}_{1} \cdot {TS}_{S} [ m ] + ϵ \qquad ( Eqn . 2 )$$

$${β}_{1} = \frac{N \cdot {\sum }_{SP} - {\sum }_{S} \cdot {\sum }_{P}}{N \cdot {\sum }_{SS} - {\sum }_{S} \cdot {\sum }_{S}} \qquad ( Eqn . 3 )$$

$${β}_{0} = \frac{{\sum }_{P} - {β}_{1} \cdot {\sum }_{P}}{N} \qquad ( Eqn . 4 )$$

$${\sum }_{S P} = {\sum }_{m = 0}^{N - 1} T {S}_{S} [ m ] \cdot {TS}_{P} [ m ] \qquad ( Eqn . 5 a )$$

$${\sum }_{S} = {\sum }_{m = 0}^{N - 1} T {S}_{S} [ m ] \qquad ( Eqn . 5 b )$$

$${\sum }_{P} = {\sum }_{m = 0}^{N - 1} T {S}_{P} [ m ] \qquad ( Eqn . 5 c )$$

TS_{P} is the timestamp of a primary (or central) sensor, and TS_{S} is the timestamp of a secondary (or peripheral) sensor. β₀ is an offset parameter, β₁ is a slope parameter, m is a timestamp index, and E is a random error term. Primary and secondary timestamps can be used for clock drift regression according to the above approach to estimate and correct clocks of the sensors. Advantageously, a high accuracy based on errors of under 1 microsecond can be achieved using this regression approach.

In alternate approaches based on a pre-calibration process, a time of flight or an estimation may be known for a message 1314 transmitted via RF means (e.g., Bluetooth) from the first sensor 1310 at T1 and received at the second sensor 1320 at T2. The second sensor 1320 may adjust its local clock timer by using the timestamp of the first sensor 1310 and T1 and the known time of flight for the message 1314. Similarly, the first sensor 1310 may adjust its local clock timer by using the timestamp of the second sensor 1320 at T3 and a known time of flight for a message 1324 received at the first sensor 1310 at T4. Messages 1314 and/or 1324 may be transmitted via RF signals (e.g., via Bluetooth). The aforementioned two-way messages may involve using both of these transmissions (each being a one-way message). While it can be assumed that the link between the sensors 1310, 1320 is symmetrical, if the times of flight of the messages 1314, 1324 are different, the adjustment of the clock timer can account for such difference.

FIG. 13A is a call flow diagram 1350 representing an example two-way exchange between a first clock timer 1311 of a first sensor 1310 and a second clock timer 1321 of a second sensor 1320. In some scenarios, at arrow 1352, a packet (e.g., message 1314) may be sent from the first sensor 1310 to the second sensor 1320 via an RF signal. At arrows 1354 and 1356, the first sensor 1310 and the second sensor 1320 respectively may capture timestamps at the first clock timer 1311 and the second clock timer 1321, which may occur simultaneously in one RF event. At arrow 1358, the second sensor 1320 may capture an event frame counter and offset from the beginning of the event, and send the offset counter to the first sensor 1310 in an acknowledgement packet at arrow 1360. Once the acknowledgement is received, the first sensor 1310 may subtract the offset from the timestamps to estimate the start of the event on the first clock timer 1311 at arrow 1362.

RF signaling between sensors can thereby enable temporal synchronization and calibration. In some implementation, any sensor can perform the calibration and clock adjustment. In some implementations, a host device can receive the timestamps from sensors, determine the adjustment(s), and send respective adjustment(s) to sensor(s). Once sensors are time synchronized, distance measurement between sensors (e.g., using ultrasound signals), and ultimately, other measurements such as of physiological characteristics of blood vessels and physiological parameters of the user such as blood pressure, can become accurate.

In an example transmission of an ultrasound signal, an ultrasound signal may be sent from the first sensor 1310 to the second sensor 1320. The first sensor 1310 can include a timestamp (such as time T1) from its local clock in the ultrasound signal. The second sensor 1320 can determine when the first sensor 1310 sent the ultrasound signal based on the timestamp included in the transmission, and when the ultrasound signal was received based on when it was received (such as time T2). Since the two clocks have already been synchronized using an approach described above, the estimated transmit time of the ultrasound signal in 1320 time domain can be derived from Eqn. 2. Based on the transmit time and the speed of sound in air or through tissue (depending on how the ultrasound signal was transmitted), a distance can be estimated between the sensors 1310, 1320 by the second sensor 1320.

Similarly, in an example ultrasound signal transmission from the second sensor 1320 to the first sensor 1310, the second sensor 1320 can include a timestamp (such as time T3) from its local clock in the ultrasound signal. The first sensor 1310 can determine when the second sensor 1320 sent the ultrasound signal based on the timestamp included in the transmission, and when the ultrasound signal was received based on when it was received (such as time T4). Hence, the estimated transmit time of the ultrasound signal can be derived from Eqn. 2. Based on the transmit time and the speed of sound in air or through tissue (depending on how the ultrasound signal was transmitted), a distance can be estimated between the sensors 1310, 1320 by the first sensor 1310.

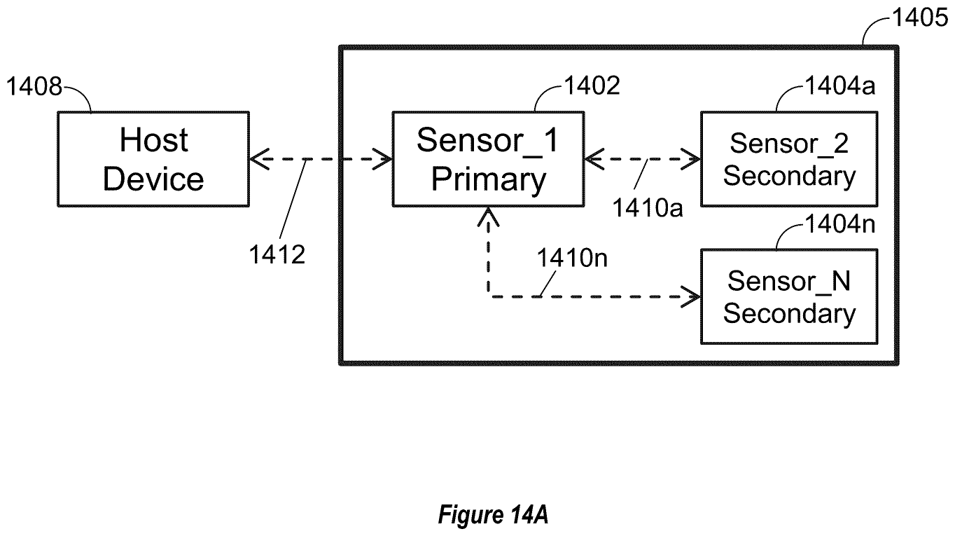

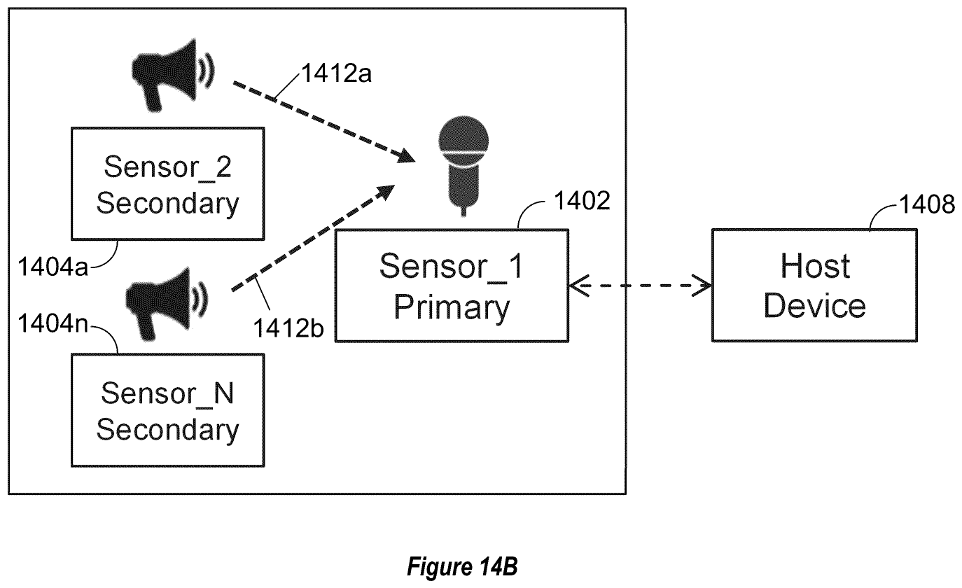

In some embodiments of the above example ultrasound signal transmissions, a MAC-level timestamp may be used to minimize delays. In some implementations, an encoding process as discussed in FIG. 4A can be used to carry the timestamp data in the ultrasound signal. In some implementations, orthogonal frequency division multiplexing (OFDM)-modulated ultrasound signals can be used with transmissions to multiple sensors, sending data (e.g., timestamp data) to sensors at different time slots. Depending on the configuration, a given sensor or a host device may determine the distance. FIGS. 12, 14A and 14B illustrate different configurations.

Referring back to the example configuration of sensors depicted in FIG. 12, sensors 1202, 1204, 1206 may each send measurements obtained via its respective sensor systems (e.g., PAPG, PPG, EKG) to the host device 1208. In some embodiments, RF signals may be used between the sensors 1202, 1204, 1206 and the host device 1208, such as via Bluetooth. In some implementations, measurements sent to the host device 1208 may include calibrated timestamps obtained by each sensor.

The host device 1208 may then calculate the distances and determine physiological characteristics of a blood vessel such as PTT and PWV based on the received timestamps, and/or physiological parameters such as blood pressure based on the PWV. A box 1205 around the sensors 1202, 1204a, 1204b and the host device 1208 represents where the measurements and determinations primarily take place (more specifically, the host device 1208). In some implementations, measurements sent to the host device 1208 may include distances between sensors measured by the sensors via ultrasound signaling. Distances in this example may include between sensors 1202 and 1204, between sensors 1202 and 1206, between sensors 1204 and 1206, or a combination thereof. In this case, the host device 1208 may determine the physiological characteristics and parameters based on the received distance(s).

FIGS. 14A and 14B are block diagrams that show another example configuration of sensors 1402, 1404a, 1404n wirelessly synchronized with a host device 1408 according to some implementations.

FIG. 14A shows a primary sensor 1402 communicatively coupled to one or more secondary sensors 1404a, 1404n via respective one or more communication links 1410a, 1410n. The primary sensor 1402 may be synchronized with the one or more secondary sensors 1404a, 1404n. In some embodiments, the primary sensor 1402 may be communicatively coupled to the host device 1408 via a communication link 1412, but secondary sensors (such as secondary sensors 1404a, 1404n) may not be directly communicatively coupled to the host device 1408. That is, one or more secondary sensors 1404a, 1404n may exchange their local timestamps with the primary sensor 1402 using one-way or two-way timestamp messages as discussed above. Timestamp exchange may occur via communication links 1410a, 1410n. Each sensor may include an RF transceiver to perform the time synchronization, and an ultrasound transceiver to perform distance measurements. In addition, the primary sensor 1402 may also include sufficient memory to store synchronized data from all secondary sensors.

In some embodiments, the primary sensor 1402 may determine physiological characteristics or parameters based on the synchronized sensor data. The primary sensor 1402 may transmit to the host device 1408, or the host device 1408 may communicate with the primary sensor 1402 only to retrieve or access, synchronized sensor data and/or the physiological characteristics or parameters via the communication link 1412. Communication links 1410a, 1410n, 1412 may be RF (e.g., Bluetooth) links. The primary sensor 1402 is therefore the main access link between the host device 1408 and all the sensors. A box 1405 around the sensors 1402, 1404a, 1404n represents where the measurements and determinations primarily take place. Contrast with the FIG. 12 example, where the host device 1208 may calculate the distances and/or physiological characteristics and parameters based on received timestamp data and/or distance data. In some implementations, however, the host device 1408 of FIG. 14 may also be configured to determine physiological characteristics or parameters based on sensor data obtained from the primary sensor 1402.

Advantageously, the FIG. 14A configuration may reduce constraints or burdens on computational resources (e.g., power, memory) or hardware capabilities on the host device 1408, which may be useful for a low-footprint host device such as a wristwatch or smartwatch (as opposed to a smartphone, laptop, tablet, etc.). It may also reduce software or algorithmic compatibility considerations between devices, as the information sent via communication link 1412 may be calculated information (e.g., by the primary sensor 1402) rather than, e.g., timestamps for synchronization with the host device.

In some configurations not shown, more than one primary sensor 1402 each exchanging timestamps with one or more secondary sensors may be communicatively coupled to the host device 1408.

FIG. 14B shows acoustic communication between time-synchronized primary sensor 1402 and one or more secondary sensors 1404a, 1404n according to some implementations. In some embodiments, the one or more secondary sensors 1404a, 1404n may send ultrasound signals via communication links 1412a, 1412n between ultrasound transmitters of the secondary sensors and ultrasound receiver of the primary sensor 1402. In some scenarios, the ultrasound signals may be transmitted over the air, where direct LOS is needed between primary sensor 1402 and a secondary sensor. In some scenarios, the ultrasound signals may be transmitted through tissue (no LOS needed between primary and secondary sensors). An ultrasound signal may contain information useful for determining distance between sensors, e.g., an identifier for the sensor sending the signal, timestamp of the sending sensor. Based on the ultrasound signals, the primary sensor 1402 may determine the distance(s) between the primary sensor 1402 and each of the one or more secondary sensors 1404a, 1404n. The distance data may be sent to or be accessible by the host device 1408. The distance data may be used to calculate physiological characteristics and parameters as mentioned above.

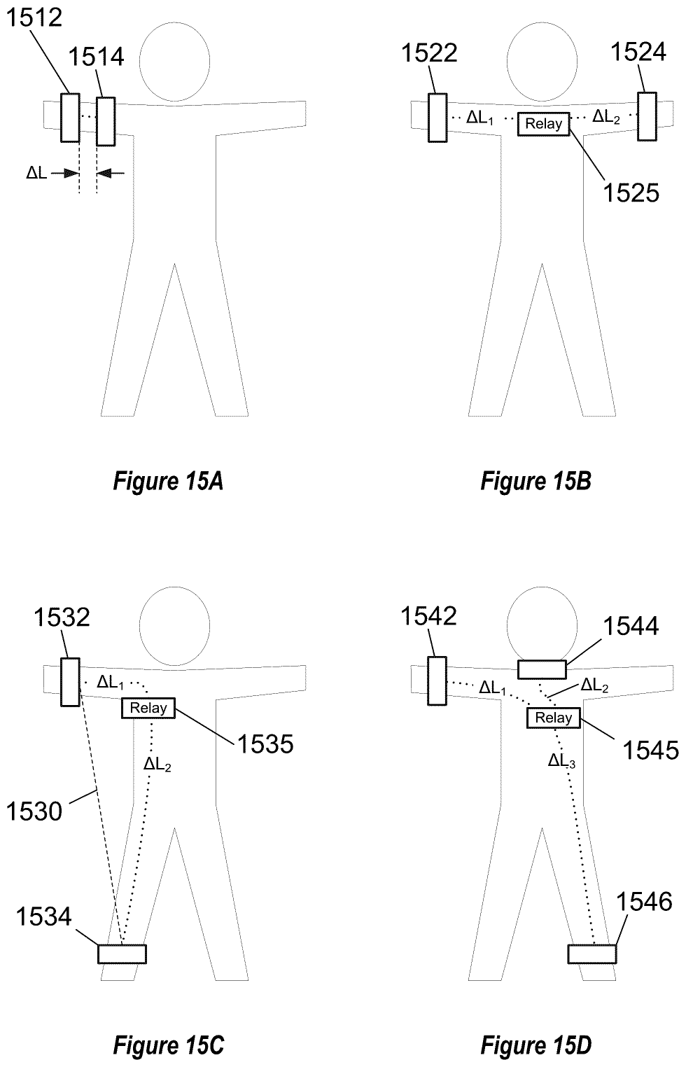

FIGS. 15A-15D illustrate various examples of distance measurements through tissue between two sensors using acoustic measurements according to some implementations. FIG. 15A is an example in which a first sensor 1512 and a second sensor 1514 are disposed on one limb. In some implementations, the distance ΔL of a blood vessel may be measured through tissue based on the speed of sound of an ultrasound signal traveling through tissue. In some implementations, there may be direct LOS between the ultrasound transceivers of the first and second sensors 1512, 1514 (e.g., above the skin), in which case, ΔL may be relatively short (along one limb) and measured based on the speed of sound of an ultrasound signal traveling through air.

Incidentally, in some scenarios, the user may be immersed at least partially in another medium, such as water. The sensors discussed herein (including but not limited to sensors 1512, 1514) may include a water detector or pressure sensor to determine that a sensor is in another medium other than air. The sensors may be hermetically sealed to prevent water from reaching sensitive components in the sensor device. Upon detecting that the sensor is in another medium, a speed of sound in such medium may be used for the calculation of the distance by the host device or the sensor, depending on whether the host device (as in the FIG. 12 example) or a sensor (as in the FIG. 14B example) is determining the distance. In such cases, the host device or the sensors may have information on speed of sound in different media.

FIG. 15B is an example in which a first sensor 1522 and a second sensor 1524 are disposed on opposite limbs. Hence, a relay sensor 1525 may be disposed between the first sensor 1522 and the second sensor 1524. The relay sensor 1525 may be an example of the relay sensor 510 of FIG. 5C. In some implementations, the distances ΔL₁ and ΔL₂ of a blood vessel may be measured through tissue based on the speed of sound of an ultrasound signal traveling through tissue, since the first and second sensors 1522, 1524 are on opposite limbs and a direct LOS over the air between the sensors may not represent the actual distance along a blood vessel in this case. That is, an ultrasound signal sent directly between the sensors 1522, 1524 may not always represent an accurate distance given the path length of a blood vessel through the body, e.g., up and down the arms. Ultrasound signals propagating through tissue may provide a better distance estimate than over the air such cases.

FIG. 15C is another example in which a first sensor 1532 and a second sensor 1534 are disposed on opposite limbs, the first sensor 1532 being on an arm and the second sensor 1534 being on a leg. Hence, a relay sensor 1535 may be disposed between the first sensor 1532 and the second sensor 1534. In some implementations, the distances ΔL₁ and ΔL₂ of a blood vessel may be measured through tissue based on the speed of sound of an ultrasound signal traveling through tissue, since, similar to the FIG. 15B, a direct LOS over the air between the sensors may not represent the actual distance along a blood vessel in this case. That is, an ultrasound signal sent via path 1530 directly between the sensors 1532, 1534 may not always represent an accurate distance given the path length of a blood vessel through the body, e.g., up the arm and down the torso.

FIG. 15D is an example in which a first sensor 1542, a second sensor 1544, and a third sensor 1546 are disposed on different places of the user's body. Hence, a relay sensor 1545 may be disposed at a location that is between the first sensor 1542 and the third sensor 1546 and between the second sensor 1544 and the third sensor 1546. In some implementations, the distance ΔL₁ of a blood vessel between the first sensor 1542 and the relay sensor 1545 may be measured based on the speed of sound or speed of air, depending on whether there is LOS between the sensors. In some implementations, the distance ΔL₂ of a blood vessel between the second sensor 1544 and the relay sensor 1545 may be measured based on the speed of sound or speed of air, depending on whether there is LOS between the sensors. In some implementations, the distance ΔL₃ of a blood vessel between the relay sensor 1545 and the third sensor 1546 may be measured based on the speed of sound or speed of air, depending on whether there is LOS between the sensors.

In each of these examples, timestamp data may be exchanged between sensors to synchronize the clock timers associated with each sensor according to approaches provided above. Such time synchronization may improve the distance measurements obtained using the ultrasound signals.

In some embodiments, a machine learning model may be used to predict a physiological parameter, e.g., blood pressure. A machine learning model may refer to a computational algorithm that indicates relationships between input variables and output variables. In some embodiments, a machine learning model can be trained. Training a machine learning model may involve, among other things, determining values of weights associated with the machine learning model, where relationships between the input variables and the output variables are based at least in part on the determined weight values. In one implementation, a machine learning model may be trained in a supervised manner using a training set that includes labeled training data. In a more particular example, the labeled training data may include inputs and manually annotated outputs that the machine learning model is to approximate using determined weight values. In another implementation, a machine learning model may be trained in an unsupervised manner in which weight values are determined without manually labeled training data.

An example training process for the machine learning model may involve providing training data that includes known photoacoustic signal data, known optical signal data, known acoustic signal data (e.g., ultrasound signal data), known EKG signal data, and/or known sensor distance data or known sensor position data, as well as the “ground truth” or the known output characteristics or parameters, e.g., PTT, PWV, blood pressure, or cardiac features (e.g., peaks) that are known. In some approaches, a portion (e.g., 20%) of the training data may be used as part of a validation set for the machine learning model. With this training data and validation set, one or more loss functions may be implemented. A loss function is an optimization function in which an error is iteratively minimized through, e.g., gradient descent. A productive learning rate that dictates the “step” the gradient descent takes when finding the lowest error may be set during training as well.

As a result, a trained machine learning model can be generated. In some implementations, such a trained machine learning model can be used to further enhance the accuracy and reliability of the estimated physiological characteristics or parameter. For example, an estimation derived from measurements from the multiple sensors and distances can be provided to the machine learning model (stored at a sensor or a host device and/or accessible by a control system thereof) to compare with a physiological characteristic of a blood vessel (e.g., PTT, PWV, heart rate) or a physiological parameter of a user (e.g., blood pressure) estimated by the machine learning model. If there is a discrepancy between the sensor-based estimation and the model-generated prediction which is greater than a threshold, the obtained estimation may be further evaluated or discarded. If discarded, the model-generated prediction may be used, or additional measurements may be taken by the sensors. In cases where there are more than two sensors on the user and a discrepancy arises between the sensor estimations and the model predictions, fewer sensors may be used as a fallback rather than all sensors. On the other hand, if the discrepancy is lower than a threshold, the sensor-based estimation may be selected or kept for further processing, sending to a host device, reporting, displaying to the user, etc.

### Example Methods

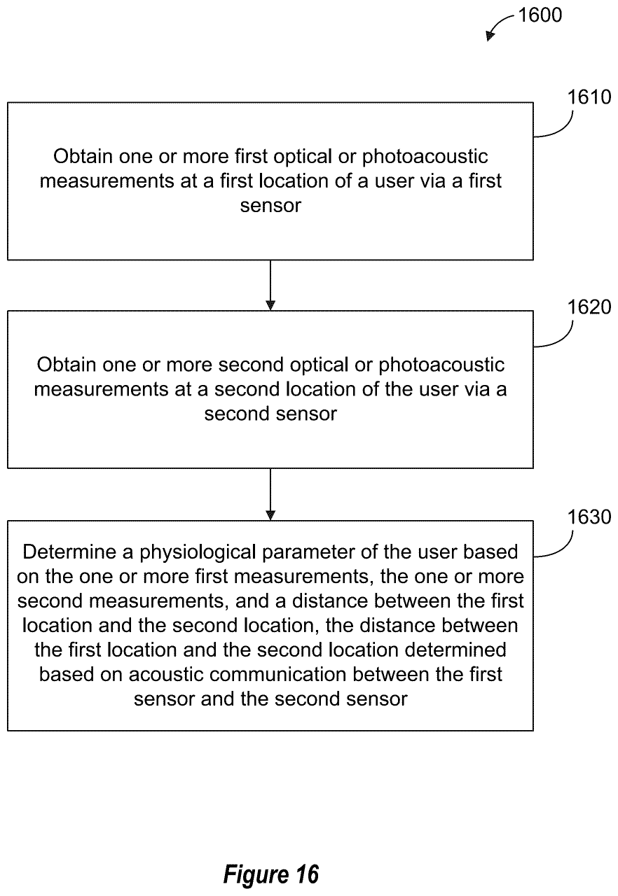

FIG. 16 is a flow diagram of a method 1600 for determining a physiological parameter of a user using synchronized sensors, according to some disclosed implementations. Structure for performing the functionality illustrated in one or more of the blocks shown in FIG. 16 may be performed by hardware and/or software components of a computerized apparatus or system (which may be implemented as a wearable device in some embodiments). Components of such apparatus or system may include, for example, one or more sensors, an acoustic transceiver system, a wireless communication system, a control system (including one or more processors), a memory, and/or a computer-readable apparatus including a storage medium storing computer-readable and/or computer-executable instructions that are configured to, when executed by the control system, cause the control system, the one or more processors, or the apparatus to perform operations represented by blocks below. Example components of the apparatus are illustrated in FIGS. 3, 4 and 5, which are described in more detail above.

The blocks of FIG. 16 may, for example, be performed by the apparatus 400 or apparatus 500 or by a similar apparatus, or a component thereof (e.g., a control system). As with other methods disclosed herein, the method outlined in FIG. 16 may include more or fewer blocks than indicated. Moreover, the blocks of methods disclosed herein are not necessarily performed in the order indicated. In some instances, one or more of the blocks shown in FIG. 16 may be performed concurrently.

At block 1610, the method 1600 may include obtaining one or more first measurements at a first location of the user via a first sensor. In some embodiments, the first sensor may include a first photoacoustic sensor or a first optical sensor. In some embodiments, the one or more first measurements may include one or more first optical measurements or one or more first photoacoustic measurements.

In some embodiments, the first sensor may be configured to obtain the first measurements comprising optical measurements, photoacoustic measurements, electrical measurements (e.g., EKG measurements), acoustic measurements, inertial measurements, or a combination thereof associated with the user at the first location.

Means for performing functionality at block 1610 may include photoacoustic sensor system 300, sensor system 401, PAPG sensor system 510, PPG sensor system 520, EKG system 540, and/or other components of the apparatus as shown in FIG. 3, 4 or 5.

At block 1620, the method 1600 may include obtaining one or more second measurements at a second location of the user via a second sensor. In some embodiments, the second sensor may include a second photoacoustic sensor or a second optical sensor. In some embodiments, the one or more second measurements may include one or more second optical measurements or one or more second photoacoustic measurements.

In some embodiments, the second sensor may be configured to obtain the second measurements comprising optical measurements, photoacoustic measurements, electrical measurements (e.g., EKG measurements), acoustic measurements, inertial measurements, or a combination thereof associated with the user at the second location.

In some embodiments, the first sensor may include a first electrode, and the second sensor may include a second electrode. In specific embodiments, the first sensor may be a first electrocardiogram (EKG) electrode, and the second sensor may be a second EKG electrode; and the one or more first measurements may include one or more first EKG measurements, and the one or more second measurements may include one or more second EKG measurements.

Means for performing functionality at block 1620 may include photoacoustic sensor system 300, sensor system 401, PAPG sensor system 510, PPG sensor system 520, EKG system 540, and/or other components of the apparatus as shown in FIG. 3, 4 or 5.

At block 1630, the method 1600 may include determining the physiological parameter of the user based on the one or more first optical or photoacoustic measurements, the one or more second optical or photoacoustic measurements, and a distance between the first location and the second location, the distance between the first location and the second location determined based on acoustic communication between the first sensor and the second sensor.

In some embodiments, the acoustic communication may include ultrasound communication; the first sensor may include an ultrasound transmitter; and the second sensor may include an ultrasound receiver configured to receive ultrasound signals. In some implementations, the first sensor may be configured to perform the acoustic communication with the second sensor via the ultrasound transmitter, and the second sensor may be configured to perform RF data communication with the host device, at least a portion of the RF data communication with the host device comprising a transmission of one or more timestamps to the host device.

In some embodiments, the second sensor may be further configured to obtain the first measurements, and perform RF data communication with a host device to transmit the first measurements, the second measurements, and the distance between the first location and the second location to the host device. In some variants, the host device may include a mobile user device. In some implementations, the mobile user device may be a wearable device. In some implementations, the first sensor and the second sensor each may be configured to perform the RF data communication with the host device via a Bluetooth protocol, at least a portion of the RF data communication including a temporal synchronization of a clock of the first sensor and a clock of the second sensor. In some implementations, the first sensor, the second sensor, or a combination thereof are configured to interface with a skin of the user via direct contact. Examples of the mobile user device may include a smartphone, wristwatch, or smartwatch. Myriad other examples are provided herein, and literally any wireless-enabled device capable of wireless communication is within the scope of this disclosure.

In some embodiments, the physiological parameter may include blood pressure of the user, the blood pressure of the user determined based on a physiological characteristic of a blood vessel of the user, the physiological characteristic of the blood vessel determined based on the first measurements obtained at the first location and the second measurements obtained at the second location. Further, the physiological characteristic of the blood vessel may include a pulse wave velocity (PWV) of the blood vessel, the PWV determined based on a pulse transmit time (PTT), the PTT determined based on a time of the first measurements associated with the user obtained at the first location and a time of the second measurements associated with the user obtained at the second location.

In some embodiments, the acoustic communication may include transmitting an ultrasound signal between the first sensor and the second sensor; and the method 1600 may further include determining a distance between the first location and the second location based at least on the ultrasound signal. In some cases, the transmitting of the ultrasound signal between the first sensor and the second sensor may occur through tissue of the user.

Means for performing functionality at block 1630 may include a control system 406 and/or other components of the apparatus as shown in FIG. 4 or 5.

In some embodiments, the method 1600 may further include synchronizing the first sensor and the second sensor using a radio frequency (RF) communication link between the first sensor and the second sensor to exchange timestamps associated with the first sensor and the second sensor, the respective timestamps configured to enable temporal synchronization between at least the first sensor and the second sensor.

In some embodiments, the method 1600 may further include obtaining one or more third measurements at a third location of the user via a third sensor; and the determining of the physiological parameter of the user may be further based at least on the one or more third measurements. In some implementations, the first sensor, the second sensor, and the third sensor each may be configured to perform the RF data communication with the host device via a Bluetooth protocol, at least a portion of the RF data communication including a temporal synchronization of a clock of the first sensor, a clock of the second sensor, and a clock of the third sensor. In some implementations, the ultrasound signal comprises a broadband signal modulated using orthogonal frequency-division multiplexing (OFDM). In some implementations, the second sensor or the host device may be configured to determine the physiological parameter associated with the user based on the first measurements, the second measurements, and the third measurements.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that may be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein, if at all, to mean “serving as an example, instance, or illustration.” Any implementation described herein as “exemplary” is not necessarily to be construed as preferred or advantageous over other implementations.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

Various modifications to the implementations described in this disclosure may be readily apparent to those skilled in the art, and the generic principles defined herein may be applied to other implementations without departing from the spirit or scope of this disclosure. Thus, the following claims are not intended to be limited to the implementations shown herein, but are to be accorded the widest scope consistent with this disclosure, the principles and the novel features disclosed herein.

Additionally, certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, various ones of the described and illustrated operations can itself include and collectively refer to a number of sub-operations. For example, each of the operations described above can itself involve the execution of a process or algorithm. Furthermore, various ones of the described and illustrated operations can be combined or performed in parallel in some implementations. Similarly, the separation of various system components in the implementations described above should not be understood as requiring such separation in all implementations. As such, other implementations are within the scope of the following claims. In some cases, the actions recited in the claims can be performed in a different order and still achieve desirable results.

Implementation examples are described in the following numbered clauses:

Clause 1: A synchronized sensor system comprising: a first sensor disposed at a first location of a user and configured to obtain first measurements associated with the user at the first location; and a second sensor disposed at a second location of the user and configured to obtain second measurements associated with the user at the second location, the second sensor configured to perform acoustic communication with the first sensor and radio frequency (RF) data communication with the first sensor, a host device, or a combination thereof; wherein at least a portion of the acoustic communication with the first sensor enables determination, by the second sensor, of a distance between the first location and the second location; and wherein at least a portion of the RF data communication enables determination, by the second sensor or the host device, of a physiological parameter associated with the user based on the first measurements associated with the user at the first location, the second measurements associated with the user at the second location, and the distance between the first location and the second location.

Clause 2: The synchronized sensor system of clause 1, wherein: the first sensor is configured to obtain the first measurements comprising optical measurements, photoacoustic measurements, electrical measurements, acoustic measurements, inertial measurements, or a combination thereof associated with the user at the first location; and the second sensor is configured to obtain the second measurements comprising optical measurements, photoacoustic measurements, electrical measurements, acoustic measurements, inertial measurements, or a combination thereof associated with the user at the second location.

Clause 3: The synchronized sensor system of any one of clauses 1-2 wherein the physiological parameter comprises blood pressure of the user, the blood pressure of the user determined based on a physiological characteristic of a blood vessel of the user, the physiological characteristic of the blood vessel determined based on the first measurements associated with the user obtained at the first location and the second measurements associated with the user obtained at the second location.

Clause 4: The synchronized sensor system of any one of clauses 1-3 wherein the physiological characteristic of the blood vessel comprises a pulse wave velocity (PWV) of the blood vessel, the PWV determined based on a pulse transmit time (PTT), the PTT determined based on a time of the first measurements associated with the user obtained at the first location and a time of the second measurements associated with the user obtained at the second location.

Clause 5: The synchronized sensor system of any one of clauses 1-4 wherein the second sensor is further configured to obtain the first measurements, and perform RF data communication with the host device to transmit the first measurements, the second measurements, and the distance between the first location and the second location to the host device.

Clause 6: The synchronized sensor system of any one of clauses 1-5 wherein the host device comprises a mobile user device.

Clause 7: The synchronized sensor system of any one of clauses 1-6 wherein the first sensor comprises a first electrode, and the second sensor comprises a second electrode; and the second sensor is further configured to derive an electrocardiogram based on signals wirelessly obtained from at least the first electrode and the second electrode.

Clause 8: The synchronized sensor system of any one of clauses 1-7 wherein the first sensor, the second sensor, or a combination thereof are configured to interface with a skin of the user via direct contact.

Clause 9: The synchronized sensor system of any one of clauses 1-8 wherein the first sensor comprises a first photoacoustic sensor or a first optical sensor; and the second sensor comprises a second photoacoustic sensor or a second optical sensor.

Clause 10: The synchronized sensor system of any one of clauses 1-9 wherein the acoustic communication comprises ultrasound communication; the first sensor comprises an ultrasound transmitter; and the second sensor comprises an ultrasound receiver configured to receive ultrasound signals.

Clause 11: The synchronized sensor system of any one of clauses 1-10 wherein the first sensor is configured to perform the acoustic communication with the second sensor via the ultrasound transmitter, and the second sensor is configured to perform the RF data communication with the host device, at least a portion of the RF data communication with the host device comprising a transmission of one or more timestamps to the host device.

Clause 12: The synchronized sensor system of any one of clauses 1-11 further comprising a third sensor disposed at a third location of the user and configured to obtain third measurements associated with the user at the third location; wherein the ultrasound signals comprise broadband signals modulated using orthogonal frequency-division multiplexing (OFDM).

Clause 13: The synchronized sensor system of any one of clauses 1-12 wherein the second sensor or the host device is configured to determine the physiological parameter associated with the user based on the first measurements, the second measurements, and the third measurements.

Clause 14: The synchronized sensor system of any one of clauses 1-13 wherein the first sensor and the second sensor are each configured to perform the RF data communication with the host device via a Bluetooth protocol, at least a portion of the RF data communication comprising a temporal synchronization of a clock of the first sensor and a clock of the second sensor.

Clause 15: The synchronized sensor system of any one of clauses 1-14 further comprising a control system, wherein the control system is configured to determine the physiological parameter of the user.

Clause 16: The synchronized sensor system of any one of clauses 1-15 wherein the control system resides in the second sensor or the host device.

Clause 17: The synchronized sensor system of any one of clauses 1-16 further comprising a data interface configured to communicate with a control system, the control system configured to determine the physiological parameter of the user based on the first measurements, the second measurements, and the distance.

Clause 18: The synchronized sensor system of any one of clauses 1-17 further comprising a third sensor disposed at a third location of the user and configured to obtain third measurements associated with the user at the third location, the third sensor comprising an ultrasound transmitter; wherein: the second sensor is further configured to perform acoustic communication with the third sensor, at least a portion of the acoustic communication with the third sensor enables determination, by the second sensor, of a distance between the second location and the third location; and the determination of the physiological parameter associated with the user is further based on the third measurements associated with the user at the third location and the distance between the second location and the third location.

Clause 19: The synchronized sensor system of any one of clauses 1-18 wherein the second sensor further comprises an ultrasound receiver configured to receive ultrasound signals, the third sensor is configured to perform the acoustic communication with the second sensor via an ultrasound transmitter, and the second sensor is configured to perform the RF data communication with the host device, at least a portion of the RF data communication with the host device comprising a transmission of one or more timestamps to the host device.

Clause 20: The synchronized sensor system of any one of clauses 1-19 wherein the first sensor, the second sensor, and the third sensor are each configured to perform the RF data communication with the host device via a Bluetooth protocol, at least a portion of the RF data communication comprising a temporal synchronization of a clock of the first sensor, a clock of the second sensor, and a clock of the third sensor.

Clause 21: A method of determining a physiological parameter of a user using synchronized sensors, the method comprising: obtaining one or more first measurements at a first location of the user via a first sensor; obtaining one or more second measurements at a second location of the user via a second sensor; and determining the physiological parameter of the user based on the one or more first measurements, the one or more second measurements, and a distance between the first location and the second location, the distance between the first location and the second location determined based on acoustic communication between the first sensor and the second sensor.

Clause 22: The method of clause 21, wherein the one or more first measurements comprise one or more first optical measurements or one or more first photoacoustic measurements, and the one or more second measurements comprise one or more second optical measurements or one or more second photoacoustic measurements.

Clause 23: The method of any one of clauses 21-22 further comprising synchronizing the first sensor and the second sensor using a radio frequency (RF) communication link between the first sensor and the second sensor to exchange respective timestamps associated with the first sensor and the second sensor, the respective timestamps configured to enable temporal synchronization between at least the first sensor and the second sensor.

Clause 24: The method of any one of clauses 21-23 wherein the acoustic communication comprises transmitting an ultrasound signal between the first sensor and the second sensor; and the method further comprises determining a distance between the first location and the second location based at least on the ultrasound signal; and the transmitting of the ultrasound signal between the first sensor and the second sensor occurs through tissue of the user.

Clause 25: The method of any one of clauses 21-24 wherein the physiological parameter comprises blood pressure of the user, the blood pressure of the user determined based on a physiological characteristic of a blood vessel of the user, the physiological characteristic of the blood vessel determined based on the first measurements obtained at the first location and the second measurements obtained at the second location; and wherein the physiological characteristic of the blood vessel comprises a pulse wave velocity (PWV) of the blood vessel, the PWV determined based on a pulse transmit time (PTT), the PTT determined based on a time of the first measurements associated with the user obtained at the first location and a time of the second measurements associated with the user obtained at the second location.

Clause 26: The method of any one of clauses 21-25 further comprising obtaining one or more third measurements at a third location of the user via a third sensor; wherein the determining of the physiological parameter of the user is further based at least on the one or more third measurements.

Clause 27: An apparatus comprising: means for obtaining one or more first measurements at a first location of a user via a first sensor; means for obtaining one or more second measurements at a second location of the user via a second sensor; and means for determining a physiological parameter of the user based on the one or more first measurements, the one or more second measurements, and a distance between the first location and the second location, the distance between the first location and the second location determined based on acoustic communication between the first sensor and the second sensor.

Clause 28: The apparatus of clause 27, wherein the apparatus comprises a host device configured to obtain, via wireless data communication with the first sensor, the one or more first measurements, the one or more second measurements, and the distance between the first location and the second location.

Clause 29: The apparatus of any one of clauses 27-28 wherein the apparatus comprises the first sensor, the first sensor configured to obtain, via wireless data communication with the first sensor, the one or more second measurements and the distance between the first location and the second location.

Clause 30: A non-transitory computer-readable apparatus comprising a storage medium, the storage medium comprising a plurality of instructions configured to, when executed by one or more processors, cause an apparatus to: obtain one or more first measurements at a first location of a user via a first sensor; obtain one or more second measurements at a second location of the user via a second sensor; and determine a physiological parameter of the user based on the one or more first measurements, the one or more second measurements, and a distance between the first location and the second location, the distance between the first location and the second location determined based on acoustic communication between the first sensor and the second sensor.

What is claimed:

1. A synchronized sensor system comprising:
a first sensor disposed at a first location of a user and configured to obtain first measurements associated with the user at the first location; and
a second sensor disposed at a second location of the user and configured to obtain second measurements associated with the user at the second location, the second sensor configured to perform acoustic communication with the first sensor and radio frequency (RF) data communication with the first sensor, a host device, or a combination thereof;
wherein at least a portion of the acoustic communication with the first sensor enables determination, by the second sensor, of a distance between the first location and the second location; and
wherein at least a portion of the RF data communication enables determination, by the second sensor or the host device, of a physiological parameter associated with the user based on the first measurements associated with the user at the first location, the second measurements associated with the user at the second location, and the distance between the first location and the second location.

2. The synchronized sensor system of claim 1, wherein:
the first sensor is configured to obtain the first measurements comprising optical measurements, photoacoustic measurements, electrical measurements, acoustic measurements, inertial measurements, or a combination thereof associated with the user at the first location; and
the second sensor is configured to obtain the second measurements comprising optical measurements, photoacoustic measurements, electrical measurements, acoustic measurements, inertial measurements, or a combination thereof associated with the user at the second location.

3. The synchronized sensor system of claim 1, wherein the physiological parameter comprises blood pressure of the user, the blood pressure of the user determined based on a physiological characteristic of a blood vessel of the user, the physiological characteristic of the blood vessel determined based on the first measurements associated with the user obtained at the first location and the second measurements associated with the user obtained at the second location.

4. The synchronized sensor system of claim 3, wherein the physiological characteristic of the blood vessel comprises a pulse wave velocity (PWV) of the blood vessel, the PWV determined based on a pulse transmit time (PTT), the PTT determined based on a time of the first measurements associated with the user obtained at the first location and a time of the second measurements associated with the user obtained at the second location.

5. The synchronized sensor system of claim 1, wherein the second sensor is further configured to obtain the first measurements, and perform RF data communication with the host device to transmit the first measurements, the second measurements, and the distance between the first location and the second location to the host device.

6. The synchronized sensor system of claim 5, wherein the host device comprises a mobile user device.

7. The synchronized sensor system of claim 1, wherein:
the first sensor comprises a first electrode, and the second sensor comprises a second electrode; and
the second sensor is further configured to derive an electrocardiogram based on signals wirelessly obtained from at least the first electrode and the second electrode.

8. The synchronized sensor system of claim 1, wherein the first sensor, the second sensor, or a combination thereof are configured to interface with a skin of the user via direct contact.

9. The synchronized sensor system of claim 1, wherein:
the first sensor comprises a first photoacoustic sensor or a first optical sensor; and
the second sensor comprises a second photoacoustic sensor or a second optical sensor.

10. The synchronized sensor system of claim 1, wherein:
the acoustic communication comprises ultrasound communication;
the first sensor comprises an ultrasound transmitter; and
the second sensor comprises an ultrasound receiver configured to receive ultrasound signals.

11. The synchronized sensor system of claim 10, wherein the first sensor is configured to perform the acoustic communication with the second sensor via the ultrasound transmitter, and the second sensor is configured to perform the RF data communication with the host device, at least a portion of the RF data communication with the host device comprising a transmission of one or more timestamps to the host device.

12. The synchronized sensor system of claim 10, further comprising a third sensor disposed at a third location of the user and configured to obtain third measurements associated with the user at the third location;
wherein the ultrasound signals comprise broadband signals modulated using orthogonal frequency-division multiplexing (OFDM).

13. The synchronized sensor system of claim 12, wherein the second sensor or the host device is configured to determine the physiological parameter associated with the user based on the first measurements, the second measurements, and the third measurements.

14. The synchronized sensor system of claim 1, wherein the first sensor and the second sensor are each configured to perform the RF data communication with the host device via a Bluetooth protocol, at least a portion of the RF data communication comprising a temporal synchronization of a clock of the first sensor and a clock of the second sensor.

15. The synchronized sensor system of claim 1, further comprising a control system, wherein the control system is configured to determine the physiological parameter of the user.

16. The synchronized sensor system of claim 15, wherein the control system resides in the second sensor or the host device.

17. The synchronized sensor system of claim 1, further comprising a data interface configured to communicate with a control system, the control system configured to determine the physiological parameter of the user based on the first measurements, the second measurements, and the distance.

18. The synchronized sensor system of claim 1, further comprising a third sensor disposed at a third location of the user and configured to obtain third measurements associated with the user at the third location, the third sensor comprising an ultrasound transmitter;
wherein:
the second sensor is further configured to perform acoustic communication with the third sensor, at least a portion of the acoustic communication with the third sensor enables determination, by the second sensor, of a distance between the second location and the third location; and
the determination of the physiological parameter associated with the user is further based on the third measurements associated with the user at the third location and the distance between the second location and the third location.

19. The synchronized sensor system of claim 18, wherein the second sensor further comprises an ultrasound receiver configured to receive ultrasound signals, the third sensor is configured to perform the acoustic communication with the second sensor via an ultrasound transmitter, and the second sensor is configured to perform the RF data communication with the host device, at least a portion of the RF data communication with the host device comprising a transmission of one or more timestamps to the host device.

20. The synchronized sensor system of claim 18, wherein the first sensor, the second sensor, and the third sensor are each configured to perform the RF data communication with the host device via a Bluetooth protocol, at least a portion of the RF data communication comprising a temporal synchronization of a clock of the first sensor, a clock of the second sensor, and a clock of the third sensor.

21. A method of determining a physiological parameter of a user using synchronized sensors, the method comprising:
obtaining one or more first measurements at a first location of the user via a first sensor;
obtaining one or more second measurements at a second location of the user via a second sensor; and
determining the physiological parameter of the user based on the one or more first measurements, the one or more second measurements, and a distance between the first location and the second location, the distance between the first location and the second location determined based on acoustic communication between the first sensor and the second sensor.

22. The method of claim 21, wherein the one or more first measurements comprise one or more first optical measurements or one or more first photoacoustic measurements, and the one or more second measurements comprise one or more second optical measurements or one or more second photoacoustic measurements.

23. The method of claim 21, further comprising synchronizing the first sensor and the second sensor using a radio frequency (RF) communication link between the first sensor and the second sensor to exchange respective timestamps associated with the first sensor and the second sensor, the respective timestamps configured to enable temporal synchronization between at least the first sensor and the second sensor.

24. The method of claim 21, wherein:
the acoustic communication comprises transmitting an ultrasound signal between the first sensor and the second sensor; and
the method further comprises determining a distance between the first location and the second location based at least on the ultrasound signal; and
the transmitting of the ultrasound signal between the first sensor and the second sensor occurs through tissue of the user.

25. The method of claim 21, wherein the physiological parameter comprises blood pressure of the user, the blood pressure of the user determined based on a physiological characteristic of a blood vessel of the user, the physiological characteristic of the blood vessel determined based on the first measurements obtained at the first location and the second measurements obtained at the second location; and
wherein the physiological characteristic of the blood vessel comprises a pulse wave velocity (PWV) of the blood vessel, the PWV determined based on a pulse transmit time (PTT), the PTT determined based on a time of the first measurements associated with the user obtained at the first location and a time of the second measurements associated with the user obtained at the second location.

26. The method of claim 21, further comprising obtaining one or more third measurements at a third location of the user via a third sensor;
wherein the determining of the physiological parameter of the user is further based at least on the one or more third measurements.

27. An apparatus comprising:
means for obtaining one or more first measurements at a first location of a user via a first sensor;
means for obtaining one or more second measurements at a second location of the user via a second sensor; and
means for determining a physiological parameter of the user based on the one or more first measurements, the one or more second measurements, and a distance between the first location and the second location, the distance between the first location and the second location determined based on acoustic communication between the first sensor and the second sensor.

28. The apparatus of claim 27, wherein the apparatus comprises a host device configured to obtain, via wireless data communication with the first sensor, the one or more first measurements, the one or more second measurements, and the distance between the first location and the second location.

29. The apparatus of claim 27, wherein the apparatus comprises the first sensor, the first sensor configured to obtain, via wireless data communication with the first sensor, the one or more second measurements and the distance between the first location and the second location.

30. A non-transitory computer-readable apparatus comprising a storage medium, the storage medium comprising a plurality of instructions configured to, when executed by one or more processors, cause an apparatus to:
obtain one or more first measurements at a first location of a user via a first sensor;
obtain one or more second measurements at a second location of the user via a second sensor; and
determine a physiological parameter of the user based on the one or more first measurements, the one or more second measurements, and a distance between the first location and the second location, the distance between the first location and the second location determined based on acoustic communication between the first sensor and the second sensor.
